(19) United States
(12) Patent Application Publication
(10) Pub. No.: US 2025/0313642 A1
(43) Pub. Date: Oct. 9, 2025

(54) METHODS OF TREATING ACUTE AND CHRONIC GRAFT VERSUS HOST DISEASE

(71) Applicant: Forte Subsidiary, Inc., Dallas, TX (US)

(72) Inventor: Paul A. WAGNER, San Juan, PR (US)

(21) Appl. No.: 19/242,082

(22) Filed: Jun. 18, 2025

Related U.S. Application Data
(63) Continuation of application No. PCT/US2023/085781, filed on Dec. 22, 2023.
(60) Provisional application No. 63/476,839, filed on Dec. 22, 2022.
(60) Provisional application No. 63/510,223, filed on Jun. 26, 2023.

(51) Int. Cl. C07K 16/28; A61K 31/519; A61K 39/395; A61P 37/06

(52) U.S. Cl. CPC C07K 16/2866; A61K 31/519; A61K 39/3955; A61P 37/06; C07K 2317/21; C07K 2317/24; C07K 2317/569; C07K 2317/622

(57) ABSTRACT

Provided herein, in certain aspects, are methods of preventing or treating acute graft versus host disease comprising administration of an IL2 receptor inhibitor, an IL15 receptor inhibitor, or an IL2/IL15 receptor inhibitor, each in combination with administration of a JAK inhibitor. Also provided herein, are methods of preventing or treating chronic graft versus host disease comprising administration of an IL2 receptor inhibitor, an IL15 receptor inhibitor, or an IL2/IL15 receptor inhibitor, each as a monotherapy.

### CROSS-REFERENCE TO RELATED APPLICATIONS

This application is a continuation application of International Application No. PCT/US2023/085781. filed December 22, 2023, which claims the benefit of U.S. provisional application Ser. No. 63/476,839 filed December 22, 2022, and U.S. provisional application Ser. No. 63/510,223 filed June 26, 2023, which are both hereby incorporated by reference in their entireties.

### TECHNICAL FIELD

Described herein are methods of preventing and/or treating acute graft versus host disease and chronic graft versus host disease.

### BACKGROUND

Interleukin receptors are involved in mediating many cellular responses including T cell immune responses. The interleukin 2 receptor is present in three forms with respect to ability to bind interleukin 2 (IL2). The low affinity form of the receptor is a monomer of the interleukin 2 receptor subunit alpha (Gene Symbol: IL2RA: also known as CD25) and is not directly involved in signal transduction. The intermediate affinity receptor form is composed of a beta/gamma subunit heterodimer, while the high affinity receptor form is composed of an alpha/beta/gamma subunit heterotrimer. Both the intermediate and high affinity forms of the receptor are involved in receptor-mediated endocytosis and transduction pathways for IL2. The IL2RB gene encodes interleukin 2 receptor subunit beta (also known as CD122). The protein encoded by the IL2RB gene (CD122) is a type I transmembrane protein with its amino-(N-)terminal domains extracellular to the plasma membrane in mature forms. CD122 protein is primarily expressed in the hematopoietic system. The IL2RG gene encodes interleukin 2 receptor subunit gamma protein (also known as CD132) which serves as the gamma subunit of IL2 receptors. Interleukin 2 receptor beta/gamma subunit heterodimers (IL2Rβ/IL2Rγ complex) are composed of CD122/CD132. Interleukin 2 receptor alpha/beta/gamma subunit heterotrimers (IL2Rα/IL2Rβ/IL2Rγ complex) are composed of CD25/CD122/CD132.

In addition to functioning in IL2-mediating signaling as the interleukin 2 receptor subunit beta. CD122 also transmits signals from the cytokine interleukin 15 (IL15). Unlike the alpha subunit of the IL2 receptor, the interleukin 15 receptor subunit alpha (Gene Symbol: IL 15RA: also known as CD215) is capable of binding its ligand (IL15) with high affinity independent of the other receptor subunits. IL15 signaling through trans-presentation of IL 15bound to the interleukin 15 receptor subunit alpha occurs to transmit signals through neighboring cells. IL15/IL15Rα bound complexes from a cell can initiate signal transduction in the trans-presentation conformation by interacting with IL15 beta/gamma receptors on neighboring cells. Interleukin 15 beta/gamma receptor heterodimers (IL15Rβ/IL15Rγ complex) have an intermediate affinity for IL15 and are composed of CD122/CD132. CD215 can also be found as part of interleukin 15 alpha/beta/gamma heterotrimers in a cis configuration for signal transduction. This IL 15 receptor alpha/beta/gamma subunit heterotrimer (IL15Rα/IL15Rβ/IL15Rγ complex) is a high affinity IL15 receptor composed of CD215/CD122/CD132 and has a binding affinity for IL15 similar to that of CD215 monomer for IL15. It is through these various interleukin receptor complexes that CD122 is involved in transmitting signals from the cytokines IL2 and IL15.

A graft versus host disease can occur after an immune-competent graft is administered to a subject and graft versus host diseases are characterized by pathogenic inflammation in organs of affected subjects. Effective prophylactics and treatments for graft versus host diseases represent significant unmet medical burdens and there is a need for improved methods of preventing and/or treating graft versus host diseases.

### SUMMARY

Cancers originating in the blood and bone marrow have the potential for long term curative treatment through hematopoietic stem cell transplantation (HSCT). HSCT can replenish immune cells and may induce anticancer beneficial effects. However, in some cases, the success of this treatment is limited by the development of graft versus host disease (GvHD). A large proportion of patients undergoing allogeneic hematopoietic stem cell transplantation (alloHSCT) go on to develop acute or chronic GvHD. Either form of GvHD is triggered by the reactivity of donor-derived immune cells against allogenic tissues in the host and remains a major unmet medical need currently with limited treatment options. Acute and/or chronic GvHD symptoms and complications often markedly affect quality of life following alloHSCT and, along with cancer reoccurrence, progression of acute GvHD or chronic GvHD is a main cause of death after alloHSCT. In addition, acute and/or chronic GvHD is responsible for approximately 15-30% of complication-related deaths following HSCT. However, the therapeutic effectiveness of alloHSCT in treating malignancies is based on the allorecognition of donor T cells, which can induce a cytotoxic effect on tumor cells from the host. This is termed a graft versus leukemia (GvL) or a graft versus tumor (GvT) effect. While allorecognition of GvL or GvT provides a therapeutic benefit to subjects in need thereof, the same phenomenon of allorecognition of non-malignant cells, tissues, and organs of the host forms the basis of graft versus host diseases. A therapy aimed at strong immunosuppression to prevent or treat acute and/or chronic GvHD has potential to abrogate the beneficial GvL or GvT effect. Conversely, it is possible that weak or no immunosuppression increases a risk or severity of acute and/or chronic GvHD following alloHSCT.

Described herein are methods of preventing chronic graft versus host disease (cGvHD) or method of treating cGvHD comprising administration of an IL2 receptor (IL2R) inhibitor to a subject in need thereof. Described herein are methods of preventing chronic graft versus host disease (cGvHD) or method of treating cGvHD comprising administration of an IL15 receptor (IL15R) inhibitor to a subject in need thereof. In some aspects, a method comprises administering to the subject an effective amount of an IL15R inhibitor, thereby preventing and/or treating cGvHD in the subject. In some embodiments, the IL2R inhibitor functions as an IL2R/IL15R inhibitor. In some embodiments, the IL15R inhibitor functions as an IL2R/IL15R inhibitor. In some embodiments, the IL2R inhibitor is a small molecule inhibitor, an antibody or its antigen-binding fragment thereof, a peptide inhibitor, or nucleotide-based inhibitor. In some embodiments, the IL 15R inhibitor is a small molecule inhibitor, an antibody or its antigen-binding fragment thereof, a peptide inhibitor, or nucleotide-based inhibitor. In some embodiments, the IL2R inhibitor is an CD122 inhibitor. In some embodiments, the IL15R inhibitor is an CD122 inhibitor. In some embodiments, the IL15R inhibitor interferes with an IL15 binding to the IL 15R. In some embodiments, the IL2R inhibitor interferes with an IL2 binding to the IL2R. In some embodiments, the IL15R inhibitor disrupts or diminishes IL15-induced signal transduction. In some embodiments, the IL15R inhibitor disrupts or diminishes CD122-mediated signal transduction. In some embodiments, the IL15R inhibitor disrupts or diminishes an IL15/IL15Ra complex from binding to an IL15Rβ/IL15Rγ complex. In some embodiments, the IL2R inhibitor disrupts or diminishes IL2-induced signal transduction. In some embodiments, the IL2R inhibitor disrupts or diminishes CD122-mediated signal transduction. In some embodiments, the IL2R inhibitor disrupts or diminishes IL2 from binding to an IL2Rβ/IL2Rγ complex. In some embodiments, the effective amount of the IL2R inhibitor. the IL15R inhibitor, or the IL2R/IL15R inhibitor does not significantly disrupt IL2 from binding to a high affinity IL2Rα/IL2Rβ/IL2Rγcomplex. In some embodiments, the effective amount of the IL2R inhibitor, the IL15R inhibitor, or the IL2R/IL15R inhibitor does not significantly disrupt IL2 from signaling through a high affinity IL2Rα/IL2Rβ/IL2Rγ complex. In some embodiments, an IL2-stimulated Janus kinase (JAK) signaling pathway is disrupted or inhibited in cells of the subject receiving the effective amount of the IL2R inhibitor. In some embodiments, an IL15-stimulated Janus kinase (JAK) signaling pathway is disrupted or inhibited in cells of the subject receiving the effective amount of the IL15R inhibitor. In some embodiments, the IL15R inhibitor is an antibody. In some embodiments, the IL2R inhibitor is an antibody. In some embodiments, the IL2R/IL15R inhibitor is an antibody. In some embodiments, the antibody is an anti-CD122 antibody. In some embodiments, the anti-CD122 antibody is a monoclonal antibody. In some embodiments, the antibody is a human antibody, a humanized antibody, or a chimeric antibody. In some embodiments, the antibody comprises an Immunoglobulin G (IgG) antibody or variant thereof. In some embodiments, the IgG antibody or variant thereof comprises an IgG1 antibody or variant thereof, an IgG2 antibody or variant thereof, an IgG3 antibody or variant thereof, or an IgG4 antibody or variant thereof. In some embodiments, the antibody or its antigen-binding fragment thereof comprises IgG-scFv, IgA, IgM, IgE antibody, nanobody, mini-antibody, minibody, scFv-CH3 KIH, Fab-scFv-Fc KIH, Fab-scFv, scFv-CH-CL-scFv, Fab′, F(ab′)2, F(ab′)3, F(ab)2-scFv2, scFv, scFv-KIH, Fab-scFy-Fc, or intrabody. In some embodiments, the anti-CD122 antibody interferes with IL15 binding to the IL15R. In some embodiments, the anti-CD122 antibody interferes with IL2 binding to the IL2R. In some embodiments, the anti-CD122 antibody interferes with IL2 binding to the IL2R and interferes with IL15 binding to the IL15R. In some embodiments, the anti-CD122 antibody diminishes or disrupts IL15-induced signal transduction. In some embodiments, the anti-CD122 antibody diminishes or disrupts IL2-induced signal transduction. In some embodiments, the administering of an effective amount of the IL15R inhibitor delays an onset of one or more symptoms of cGvHD in the subject. In some embodiments, the administering an effective amount of the IL15R inhibitor or the IL2R inhibitor alleviates one or more symptoms of cGvHD in the subject. In some embodiments, the administering an effective amount of the IL15R inhibitor or the IL2R inhibitor significantly relieves cGvHD severity in the subject. In some embodiments, the administering improves an objective response rate (ORR) at 6 months compared to a first-line standard of care therapy, a second-line standard of care therapy, or a third-line standard of care therapy. In some embodiments, the first-line standard of care therapy comprises treatment with one or more corticosteroids. In some embodiments, the first-line standard of care therapy comprises treatment with one or more JAK inhibitors. In some embodiments, the first-line standard of care therapy comprises treatment with ruxolitinib. In some embodiments, the second-line standard of care therapy comprises treatment with a JAK inhibitor. In some embodiments, the second-line standard of care therapy comprises treatment with either ruxolitinib or ibrutinib. In some embodiments, the second-line standard of care therapy comprises treatment with one or more corticosteroids. In some embodiments, the third-line standard of care therapy comprises treatment with belumosudil. In some embodiments, the one or more symptoms of cGvHD comprises skin rash, raised skin, discolored skin, itchy skin, thickened skin, tightened skin, damaged sweat glands, intolerance to temperature changes, abdominal swelling, yellow discoloration of the eyes, jaundice, elevated or abnormal liver enzyme levels in the blood, dry eyes, changes in vision, dry mouth, white patches in the oral cavity, painful mouth ulcers, pain or sensitivity to hot, cold, spicy, and/or acidic foods, pain or sensitivity to carbonated beverages, shortness of breath, dry cough, chronic cough, wheezing, difficulty breathing, pulmonary changes observed on a chest X-ray, difficulty swallowing, difficulty eating, pain with swallowing, gum disease, tooth decay, loss of appetite, weight loss, nausea, vomiting, diarrhea, stomach pain, fatigue, muscle weakness, muscle cramps, neuromuscular pain, decreased range of motion in joints, decreased range of extension of fingers, wrists, elbows, knees, and/or ankles, tightness in joints or in connective tissue, change in physical activity level, change in locomotor activity level, change in posture, change in gait, vaginal dryness, vaginal itching, vaginal pain, vaginal ulcerations and scarring, narrowing of the vagina, painful vaginal intercourse, narrowing and/or scaring of the urethra, itching and/or scarring of the penis and scrotum, irritation of the penis, change in skin texture, change in skin integrity, scaling of skin, areas of denuded skin, loss of hair on the head, hard nails, brittle nails, nail loss, changes in nail texture, premature graving of the hair, or changes in hair texture, or any combination thereof. In some embodiments, the administering an effective amount of the IL15R inhibitor or the IL2R inhibitor reduces expression of one or more biomarkers of cGvHD in the subject. In some embodiments, the administering an effective amount of the IL15R inhibitor or the IL2R inhibitor increases the survival rate of the subject. In some embodiments, the administering of an effective amount of the IL15R inhibitor or the IL2R inhibitor decreases the risk of cGvHD-symptom relapse. In some embodiments, the IL15R inhibitor or the IL2R inhibitor is administered systemically, locally, intradermally, subcutaneously, or topically. In some embodiments, the IL15R inhibitor or IL2R inhibitor administered systemically is administered by intravenous injection, by enteral administration, or through inhalation. In some embodiments, the IL15R inhibitor or IL2R inhibitor administered by enteral administration is administered orally.

Described herein are methods of preventing acute and/or chronic GvHD or treating acute and/or chronic GvHD comprising administering an effective amount of i) an IL15R inhibitor and ii) a JAK inhibitor, to a subject in need thereof. Also described herein are methods of preventing acute and/or chronic GvHD or treating acute and/or chronic GvHD comprising administering an effective amount of i) an IL2R inhibitor and ii) a JAK inhibitor, to a subject in need thereof. In some aspects, a method comprises administering to the subject an effective amount of: i) an IL15R inhibitor and a JAK inhibitor, or ii) an IL2R inhibitor and a JAK inhibitor, thereby preventing and/or treating GvHD in the subject. In some embodiments, the IL15R inhibitor functions as an IL2R/IL15R inhibitor. In some embodiments, the IL2R inhibitor functions as an IL2R/IL15R inhibitor. In some embodiments, the GvHD is acute graft versus host disease (aGvHD). In some embodiments, the aGvHD is steroid refractory aGvHD. In some embodiments, the aGvHD is JAK inhibitor refractory aGvHD. In some embodiments, the aGvHD is ruxolitinib refractory aGvHD. In some embodiments, the GvHD is chronic graft versus host disease (cGvHD). In some embodiments, the IL15R inhibitor and the JAK inhibitor are co-administered. In some embodiments, the IL2R inhibitor and the JAK inhibitor are co-administered. In some embodiments, the IL15R inhibitor and JAK inhibitor or the IL2R inhibitor and JAK inhibitor are administered separately or sequentially. In some embodiments, the JAK inhibitor is selected from the group consisting of abrocitinib, baricitinib, delgocitinib, fedratinib, filgotinib, oclacitinib, pacritinib, peficitinib, ruxolitinib, tofacitinib, itacitinib and upadacitinib. In some embodiments, the JAK inhibitor is ruxolitinib. In some embodiments, the IL15R inhibitor is a CD122 inhibitor. In some embodiments, the IL2R inhibitor is a CD122 inhibitor. In some embodiments, the IL15R inhibitor is an antibody or its antigen-binding fragment thereof, a small molecule inhibitor, a peptide inhibitor, or a nucleotide-based inhibitor. In some embodiments, the IL2R inhibitor is an antibody or its antigen-binding fragment thereof. a small molecule inhibitor, a peptide inhibitor, or a nucleotide-based inhibitor. In some embodiments, the antibody is a human antibody, a humanized antibody, or a chimeric antibody. In some embodiments, the antibody comprises an Immunoglobulin G (IgG) antibody or variant thereof. In some embodiments, the IgG antibody or variant thereof comprises an IgGI antibody or variant thereof, an IgG2 antibody or variant thereof, an IgG3 antibody or variant thereof, or an IgG4 antibody or variant thereof. In some embodiments, the antibody or its antigen-binding fragment thereof comprises IgG-scFv, IgA, IgM, IgE antibody, nanobody, mini-antibody, minibody, scFv-CH3 KIH. Fab-scFy-Fc KIH, Fab-scFv, scFv-CH-CL-scFv, Fab', F(ab′)2, F(ab′)3,_F(ab′)2-scFv2, scFv, scFv-KIH, Fab-scFy-Fc, or intrabody. In some embodiments, the IL15 receptor inhibitor is an anti-CD122 antibody. In some embodiments, the IL2 receptor inhibitor is an anti-CD122 antibody. In some embodiments, the JAK inhibitor is administered first and the IL15R inhibitor is administered second. In some embodiments, the JAK inhibitor is administered first and the IL2R inhibitor is administered second. In some embodiments, the administering of an effective amount of the IL15R inhibitor and JAK inhibitor or an effective amount of the IL2R inhibitor and JAK inhibitor delays an onset of one or more symptoms of aGvHD or cGvHD in the subject. In some embodiments, the administering of an effective amount of the IL15R inhibitor and JAK inhibitor or the administering of an effective amount of the IL2R inhibitor and JAK inhibitor alleviates one or more symptoms of aGvHD or cGvHD in the subject. In some embodiments, the one or more symptoms of aGvHD comprises itchy skin, skin rash, reddened patches on the skin, yellow discoloration of the skin, blisters on the skin, exposed surfaces of the skin flaking off, yellow discoloration of the eyes, jaundice, elevated liver enzyme levels in the blood, nausea, vomiting, diarrhea, abdominal cramping, loss of appetite, or weight loss, or any combination thereof. In some embodiments, the one or more symptoms of cGvHD comprises skin rash, raised skin, discolored skin, itchy skin, thickened skin, tightened skin, damaged sweat glands, intolerance to temperature changes, abdominal swelling, yellow discoloration of the eyes, jaundice, elevated or abnormal liver enzyme levels in the blood, dry eyes, changes in vision, dry mouth, white patches in the oral cavity, painful mouth ulcers, pain or sensitivity to hot, cold, spicy, and/or acidic foods, pain or sensitivity to carbonated beverages, shortness of breath, dry cough, chronic cough, wheezing, difficulty breathing, pulmonary changes observed on a chest X-ray, difficulty swallowing, difficulty eating, pain with swallowing, gum disease, tooth decay, loss of appetite, weight loss, nausea, vomiting, diarrhea, stomach pain, fatigue, muscle weakness, muscle cramps, neuromuscular pain, decreased range of motion in joints, decreased range of extension of fingers, wrists, elbows, knees, and/or ankles, tightness in joints or in connective tissue, change in physical activity level, change in locomotor activity level, change in posture, change in gait, vaginal dryness, vaginal itching, vaginal pain, vaginal ulcerations and scarring, narrowing of the vagina, painful vaginal intercourse, narrowing and/or scaring of the urethra, itching and/or scarring of the penis and scrotum, irritation of the penis, change in skin texture, change in skin integrity. scaling of skin, areas of denuded skin, loss of hair on the head, hard nails, brittle nails, nail loss. changes in nail texture, premature graving of the hair, or changes in hair texture, or any combination thereof. In some embodiments, the administering of an effective amount of the IL15R inhibitor and JAK inhibitor or the administering of an effective amount of the IL2R inhibitor and JAK inhibitor reduces expression of one or more biomarkers of aGvHD or cGvHD in the subject. In some embodiments, the administering of an effective amount of the IL15R inhibitor and JAK inhibitor or the administering of an effective amount of the IL2R inhibitor and JAK inhibitor increases the survival rate of the subject. In some embodiments, the administering of an effective amount of the IL15R inhibitor and JAK inhibitor or the administering of an effective amount of the IL2R inhibitor and JAK inhibitor increases the survival rate of the subject compared with a subject treated with a JAK inhibitor as a monotherapy. In some embodiments, the administering of an effective amount of the IL15R inhibitor and JAK inhibitor or the administering of an effective amount of the IL2R inhibitor and JAK inhibitor decreases a risk of aGvHD-symptom relapse or cGvHD-symptom relapse. In some embodiments, the IL15R inhibitor and JAK inhibitor or the IL2R inhibitor and JAK inhibitor are administered by the same route of administration. In some embodiments, the IL15R inhibitor and JAK inhibitor or the IL2R inhibitor and JAK inhibitor are administered by separate routes of administration. In some embodiments, the IL15R inhibitor or the IL2R inhibitor is administered systemically, locally, intradermally, subcutaneously, or topically, and the JAK inhibitor is administered systemically, locally, intradermally, subcutaneously, or topically. In some embodiments, the IL15R inhibitor or IL2R inhibitor administered systemically is administered by intravenous injection, by subcutaneous injection, by enteral administration, or through inhalation. In some embodiments, the JAK inhibitor administered systemically is administered by intravenous injection, by subcutaneous injection, by enteral administration, or through inhalation. In some embodiments, the IL15R inhibitor or IL2R inhibitor administered by enteral administration is administered orally. In some embodiments, the JAK inhibitor administered by enteral administration is administered orally. In some embodiments, the administering an effective amount of the IL15R inhibitor and JAK inhibitor or the administering an effective amount of the IL2R inhibitor and JAK inhibitor significantly prevents development of an extent of cGvHD severity in the subject. In some embodiments, the administering an effective amount of the IL15R inhibitor and JAK inhibitor or the administering an effective amount of the IL2R inhibitor and JAK inhibitor significantly relieves GvHD severity. In some embodiments, the administering improves an ORR at 28 days compared to treatment comprising ruxolitinib monotherapy. In some embodiments, the administering significantly prevents development of an extent of cGvHD severity in the subject according to a total cGvHD assessment compared to placebo treatment, a first-line standard of care cGvHD therapy, a second-line standard of care cGvHD therapy, or a third-line standard of care cGvHD therapy. In some embodiments, the first-line standard of care therapy comprises treatment with one or more corticosteroids. In some embodiments, the first-line standard of care therapy comprises treatment with one or more JAK inhibitors. In some embodiments, the first-line standard of care therapy comprises treatment with ruxolitinib. In some embodiments, the second-line standard of care therapy comprises treatment with a JAK inhibitor. In some embodiments, the second-line standard of care therapy comprises treatment with either ruxolitinib or ibrutinib. In some embodiments, the second-line standard of care therapy comprises treatment with one or more corticosteroids. In some embodiments, the third-line standard of care therapy comprises treatment with belumosudil. In some embodiments, the administering significantly prevents development of an extent of cGvHD severity in the subject according to a total cGvHD assessment compared to treatment comprising ruxolitinib monotherapy.

In some aspects described herein are methods of preventing or treating chronic graft versus host disease (cGvHD) in a subject in need thereof, the methods comprising: administering to the subject an effective amount of an IL2R/IL15R inhibitor, thereby preventing or treating cGvHD in the subject.

In some aspects described herein are methods of preventing or treating chronic graft versus host disease (cGvHD) in a subject in need thereof, the methods comprising: administering to the subject an effective amount of: i) an IL2R inhibitor, ii) an IL15R inhibitor, or iii) an IL2R/IL15R inhibitor, thereby preventing or treating cGvHD in the subject.

In some aspects described herein are methods of preventing or treating GvHD in a subject in need thereof, the method comprising: administering to the subject an effective amount of i) an IL2 receptor (IL2R) inhibitor, an IL15 receptor (IL15R) inhibitor, or an IL2R/IL15R inhibitor, and ii) a JAK inhibitor, thereby preventing or treating GvHD in the subject.

In some aspects described herein the effective amount of the IL2R inhibitor, the IL15R inhibitor, or the IL2R/IL15R inhibitor is a therapeutically effective amount to treat one of more symptoms of aGvHD or cGvHD in the subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-FIG. 1B show graphs and IC₅₀ calculations for anti-CD122 antibody inhibition of IL2 and IL15 signaling. In FIG. 1A, results are graphed and IC₅₀ calculations for anti-CD122 antibodies are shown. In vitro testing, in which a cell line expressing the intermediate affinity IL-βγ receptor using a reporter assay expressing luciferase under IL2 stimulation, was used. In FIG. 1B, results are graphed and IC₅₀ calculations for anti-CD122 antibodies are shown. In vitro testing, in which a cell line expressing the intermediate affinity IL-βγ receptor using a reporter assay expressing luciferase under IL15 stimulation, was used.

FIG. 2 shows a graph for anti-CD122 antibody (Antibody 1 and Antibody 2) inhibition of IL2 signaling in a cell line expressing the high affinity IL-αβγ receptor using a reporter assay.

FIG. 3 shows an acute GvHD study design for a dose-ranging investigation of anti-CD122 antibodies.

FIG. 4 shows an acute GvHD study design for single therapy and combination therapy treatment test groups.

FIG. 5 shows a graph of survival duration in an acute GvHD mouse model comparing effectiveness of anti-CD122 antibody (Antibody 1) plus ruxolitinib treatment versus ruxolitinib monotherapy versus vehicle-treated control.

FIG. 6 shows an allogeneic chronic GvHD study design for assessing anti-CD122 antibody monotherapy treatment effectiveness versus ruxolitinib monotherapy treatment effectiveness versus vehicle-treated control.

FIG. 7 shows a graph of GvHD scores in a chronic GvHD mouse model comparing anti-CD122 antibody (Antibody 3) monotherapy versus JAK inhibitor (ruxolitinib) monotherapy versus vehicle-treated control.

### DETAILED DESCRIPTION

Immune cell responses are often context dependent and in some cases are influenced by signals from their environment through a variety of receptor-ligand interactions. For instance, in various embodiments, these signals amplify and modify a T cell receptor (TCR) signal received by antigenic stimulation in a resting naïve or memory T cell, regulate T cell proliferation and differentiation in recently activated T cells, or control effector functions in particular somatic environments. IL2 and IL15 share similar and contrasting roles in regulation of T cell function. As a non-limiting example, both IL2 and IL15 are involved in T cell differentiation. IL2 promotes the differentiation of immature T cells into regulatory T cells, which thereby are capable of suppressing other T cells that could attack normal healthy cells in the body. IL2 signaling is involved in peripheral tolerance through the elimination of self-reactive T cells by way of the activation-induced cell death (AICD) pathway. In some cases. IL2 also promotes the differentiation of immature T cells into either effector T cells or into memory T cells when an initial T cell is stimulated by an antigen. IL2 has also been demonstrated to enhance the activity of both cytotoxic T cells and natural killer (NK) cells. In some cases. IL15 regulates the activation and proliferation of T cells and NK cells. In contrast to IL2 signaling, in some cases, IL15 signaling inhibits IL2-mediated AICD by eliciting antiapoptotic actions. In some instances, IL15 stimulates the persistence of memory phenotype CD8+ T cells that are involved in the elimination of invading pathogens, thereby protecting the subject against infection.

IL2 and IL15 have distinct means for initiating signaling through their receptors. IL2 is a predominantly secreted cytokine that either in its soluble form or linked to extracellular matrix can bind to heterodimeric (IL2Rβ/IL2Rγ) and heterotrimeric (IL2Rα/IL2Rβ/IL2Rγ) receptor complexes both involving CD122 on the surface of activated cells. IL15, mainly secreted along with IL15Rα, is primarily membrane bound and induces signaling in the context of cell-cell contacts, at the immunological synapse. IL15Rα presents membrane bound IL15 in the trans configuration to neighboring CD8+ T cells and NK cells. Despite these differences in the mechanisms of the initiation of ligand-mediated signaling, once activated. IL2 receptor complexes and IL15 receptor complexes activate shared molecular pathways including the JAK1/JAK3/STAT5, the PI3K, and the MAPK signal transduction pathways. IL2Rβ binds to JAKI and IL2Rγ binds to JAK3. Ligand binding of IL2R and IL15R can result in activation of JAK kinases and phosphory lation of tyrosine residues on IL2Rβ and IL2Rγ. Tyrosine phosphorylation of IL2Rβ permits recruitment of STAT5A, STAT5B, STAT3, and/or SHC1. Following their recruitment to IL2R or IL15R, STAT5 and/or SHC1 proteins can be phosphorylated by one or more JAK proteins. Tyrosine phosphorylation of STAT5 permits protein dimerization, subsequent nuclear translocation and STAT5-mediated gene transcription. Tyrosine phosphorylation of SHC1 permits recruitment of GRB2 and SOS to facilitate activation of the Raf-ERK MAP kinase signaling cascade. In some cases, activation of these pathways modulates gene transcription to regulate apoptosis, proliferation, or differentiation of immune cells. Functioning in both IL2 receptors and IL15 receptors, CD122 serves critical roles in these various capacities.

### Graft Versus Host Disease (GvHD)

Graft versus host disease (GvHD) is a condition which often occurs following an allogeneic transplant. In GvHD, donated multipotent hematopoietic stem cells (typically derived from bone marrow, peripheral blood stem cells, umbilical cord stem cells, or stem cells of other sources) recognize the recipient's cells and organs as foreign, and these donated cells and their derivatives attack the host's body. There are two recognized forms of GvHD: acute graft versus host disease (aGvHD) and chronic graft versus host disease (cGvHD). A host subject receiving an allogeneic transplant is at risk for developing aGvHD, cGvHD, or both conditions. Acute GvHD and chronic GvHD may affect the skin, the gastrointestinal (GI) tract, the liver, and other tissues and organs. Pathogenic inflammation can occur in various affected organs in subjects presenting with aGvHD and/or cGvHD.

Acute GvHD occurs in up to 50% of transplant recipients with onset typically occurring within 3 months of transplant. Moderate-to-severe aGvHD develops in about 20-50% of recipients of an HLA-identical sibling allogeneic stem cell transplant. Estimates of mortality directly attributable to aGvHD or treatment thereof occurs in approximately 10-20% of patients. In aGvHD, a combination of symptoms in various organs is often involved including skin (rash), GI tract (vomiting and/or diarrhea), and liver (e.g., jaundice). The skin is the most commonly affected site in aGvHD and symptoms often manifest as a rash resembling a sunburn with blistering or peeling and often affect the back, shoulders, ears, neck, palms of hands, and soles of feet of the host. In some patients, the most common manifestation of aGvHD is a maculopapular rash, typically occurring at or near the time of white blood cell engraftment. This rash typically involves the nape of the neck, ears, shoulders, palms of the hands, and soles of the feet initially. The rash may later spread to involve the entire integument. Histologic examination of the skin often reveals changes in both epidermal and dermal layers. Characteristic findings of aGvHD pathology in the skin include exocytosed lymphocytes, dyskeratotic epidermal keratinocytes, follicular involvement, satellite lymphocytes near dyskerototic epidermal keratinocytes, and dermal perivascular lymphocytic infiltration. A consistent aGvHD pathological feature in the skin is apoptosis at the base of crypts.

aGvHD in the GI tract often causes abdominal pain, diarrhea, persistent nausea and/or vomiting, and a loss of appetite or a feeling of satiety after eating a small amount of food. A diagnosis of GI involvement in aGvHD may require pathological evaluation of biopsied tissue. An extent of GI involvement in aGvHD can be measured according to volume of diarrhea in the subject per day.

In some cases, aGvHD also affects the liver, causing symptoms such as dark urine. jaundice, and elevated liver enzymes in the blood. An extent of liver involvement in aGvHD can be measured according to serum total bilirubin levels in a patient and also if bilirubin levels in the patient rise over time.

The stage of liver involvement in aGvHD can be combined with assessments of the stage of cutaneous and GI tract involvement to determine an overall severity grade for aGvHD. Less commonly involved in aGvHD are the hematopoietic system, eyes, lungs, and/or kidneys. Pathological alterations in these organ systems are not used to establish an initial diagnosis of aGvHD, but may be informative of overall severity grade for aGvHD once aGvHD diagnosis has already been established. Hematopoietic involvement in aGvHD can manifest as thymic atrophy, a cytopenia (e.g., thrombocytopenia), and/or hypogammaglobulinemia (e.g., IgA deficiency). Involvement in the eyes in aGvHD can lead to photophobia, hemorrhagic conjunctivitis, and an inability to completely close the eyes. Kidney involvement in aGvHD can present as nephritis or nephrotic syndrome. Lung involvement in aGvHD can manifest itself as interstitial pneumonitis. A diagnosis of aGvHD can be made on clinical grounds alone in a subject that presents with a typical aGvHD rash, abdominal cramps with diarrhea, and serum bilirubin concentrations that rise within the first 100 days following transplantation.

Occurrence of prior aGvHD is a main risk factor for development of cGvHD. The pathogenesis of cGvHD is complex and includes tissue damage, unusual antigen presentation and aberrant myeloid and lymphoid interactions. The initial phase of cGvHD includes an effect of early post-transplant inflammation and tissue injury. Excessive release of inflammatory cytokines activates antigen-presenting cells which stimulate the activation of donor alloreactive T cells having enhanced T cell effector lineages. Macrophages are also sequestered in affected tissues. Following this initial phase, in some cases, cGvHD progresses to the presence of chronic inflammation and dysregulation of the immune system operating outside of the normal regulatory immune responses. Further progression of cGvHD is evident as aberrant repair mechanisms lead to a release of profibrotic mediators via monocytes and macrophages. In some cases, this causes fibroblast activation, collagen deposition, and ultimately fibrosis.

Symptoms of cGvHD in the skin can include rash, raised, or discolored skin areas, and skin thickening or tightening. Signs of cGvHD in the liver include abdominal swelling, a yellow discoloration of the eyes and or skin (jaundice), and abnormal blood test results including elevated liver enzymes. Signs of cGvHD in the eyes include dry eyes or changes in vision. Signs of cGvHD in the mouth and oral region include dry mouth, white patches on the inside of the mouth, and pain or sensitivity to spicy foods. Signs of pulmonary cGvHD include shortness of breath, dry cough, or alterations seen on a chest X-ray. Signs of cGvHD in the GI tract include difficulty swallowing, pain with swallowing, or weight loss. Signs of neuromuscular cGvHD include fatigue, or muscle weakness or pain. In some cases, cGvHD affects the vagina or vulva resulting in vaginal dryness or pain, cGvHD affecting the connective tissue often results in tightness in the joints and a decreased range of bodily motion. Chronic GvHD develops in up to 40% of transplant recipients and onset typically occurs after about 100 days following transplant. In addition to involvement of the skin, GI tract, and liver, cGvHD symptoms may involve dysfunction in the lungs, mucosal surfaces (e.g., eyes, mouth, and/or GI tract), muscles, and joints (e.g., connective tissues).

In some cases, GvHD is diagnosed during a physical examination by a medical practitioner by observation of GvHD-related symptoms and/or by evaluating the results of biopsies and clinical lab tests. In the case of cGvHD, symptoms sometimes present as vague or even transitory which may make a diagnosis of cGvHD possible only following the exclusion of other potential causes of symptomatology. Although the manifestation of cGvHD symptoms can be heterogeneous at onset, certain features are termed diagnostic features sufficient to establish a diagnosis of cGvHD. Diagnostic features of cGvHD include sclerosis, lichen-planus-like lesions, poikiloderma, esophageal webs, and fasciitis and bronchiolitis obliterans. In contrast, distinctive features which are highly suggestive of cGvHD but are not sufficient by themselves to establish diagnosis include oral ulcers and atrophy, onchodystrophy, and sicca syndrome. Distinctive features of cGvHD such as those listed above may be confirmed as cGvHD through biopsy or by other diagnostic test criteria. The most frequent sites of pathology involved at the initial diagnosis of cGvHD are skin, mouth (e.g., lichen-planus-like lacy buccal involvement, xerostomia from salivary gland dysfunction, food sensitivity, oral pain, erythema, and/or non-healing mouth ulcers), liver, and eye. Less frequently involved sites of pathology at the initial diagnosis of cGvHD are GI tract (e.g., as evidenced by unexplained weight loss), lung, esophagus, female genital tract, and joints.

To attempt to mitigate the risk of GvHD occurrence, the best HLA-matched donor is selected for the transplant into the host. Additionally, prophylactic (preventative) treatments often aimed at suppressing the immune system are regularly initiated following transplant. These treatments are aimed at decreasing the ability of the donor's cells and derivatives thereof for initiating an active immune response against host cells, tissues, and organs. Fungal, bacterial, and viral infections are major risks for subjects undergoing an immunosuppressive prophylactic treatment regimen as the host's body will maintain a decreased ability to fight infection while under immunosuppression. Prophylactic antibiotics, antifungals and antiviral medicines are often administered during immunosuppressive therapy to decrease risks of infection.

Treatments currently used in an attempt to ameliorate symptoms in aGvHD include administration of corticosteroids, ruxolitinib, sirolimus, mycophenolate mofetil (CellCept). mycophenolate sodium (Myfortic), or antithymocyte globulin. These drugs may be administered orally and/or intravenously. A different therapeutic approach to treat aGvHD is extracorporeal photopheresis, involving removal and separation of leukocytes from the affected subject and then exposure of those cells to ultraviolet irradiation in the presence of a photosensitizing agent prior to reinfusion of the treated cells into the subject. TNFα inhibitors (e.g., adalimumab or infliximab) have been attempted as therapeutics to treat or ameliorate symptoms of aGvHD. In a first-line standard of care therapy including treatment using one or more corticosteroids, approximately 30-50% of patients have an inadequate response to treatment. In patients with grade 3 and grade 4 aGvHD disease, following solely a first-line standard of care therapy leads to a 2-year mortality rate of >70%.

In contrast, treatments currently used to attempt to reduce symptoms of cGvHD include various forms of immunosuppressive therapies. In addition, the heterogenous nature of cGvHD has led to a variety of treatments that can be tried, such as extracorporeal photopheresis. although very few of which are approved by the FDA to treat the condition. Approved treatments include, ruxolitinib, belumosudil, and ibrutinib. A first-line standard of care therapy for cGvHD includes treatment with one or more corticosteroids, however there is limited success following this first-line therapy. Approximately 50-60% of patients undergoing corticosteroid treatment for cGvHD will require initiation of a second-line therapy within 2 years.

In various aspects of methods provided herein aGvHD typically occurs in the early post-transplantation period. The initial signs and symptoms often occur during the time of white blood cell engraftment. Although initial definitions of aGvHD required onset of symptoms before 100 days post transplantation, the current consensus uses clinical findings rather than a set time period to differentiate aGvHD from cGvHD. The skin, gastrointestinal tract, and liver are the principal target organs for aGvHD. Other affected organs include the hematopoietic system, the eyes, kidneys, and lungs. Diagnosis of aGvHD is often made in post-hematopoietic cell transplantation in a patient having a rash, abdominal cramps with diarrhea, and rising serum bilirubin concentration during the first 100 days following transplantation. However, histologic confirmation via skin and/or gastrointestinal biopsy is often undertaken. Biomarkers for aGvHD include suppression of tumorigenicity 2 (ST2), regenerating islet-derived 3-alpha (REG3alpha). and tumor necrosis factor receptor 1 (TNFR1).

In contrast, cGvHD presents with a variety of clinical features that often resemble autoimmune and other immunologic disorders, such as scleroderma. Sjogren's syndrome. primary biliary cirrhosis, and bronchiolitis obliterans. In some cases, clinical manifestation is widespread. Alternatively, symptoms are restricted to a single organ or site. The most frequent symptoms include skin involvement (resembling lichen planus or cutaneous scleroderma), dry oral mucosa, gastrointestinal tract ulcerations and sclerosis, elevated serum bilirubin, and bronchiolitis obliterans. Promising serum biomarkers for cGvHD include CXCL9, ST2, matrix metalloproteinase-3, osteopontin. CXCL10, CXCL11, and CD163.

### cGvHD Treatment With IL2R Inhibitor, IL15R Inhibitor, or IL2R/IL15R Inhibitor

Disclosed herein, in certain aspects, are IL2 receptor (IL2R) inhibitors for use in methods of preventing and/or treating chronic graft versus host disease. Disclosed herein, in certain aspects, are IL15 receptor (IL15R) inhibitors for use in methods of preventing and/or treating chronic graft versus host disease. Disclosed herein, in certain aspects, are IL2R/IL15R inhibitors for use in methods of preventing and/or treating chronic graft versus host disease. Methods for prophylaxis of cGvHD or treatment for cGvHD comprising administration of an IL2R inhibitor are described herein. Methods for prophylaxis of cGvHD or treatment for cGvHD comprising administration of an IL15R inhibitor are described herein. Methods for prophylaxis of cGvHD or treatment for cGvHD comprising administration of an IL2R/IL15R inhibitor are described herein. In some embodiments, the IL2R inhibitor is a small molecule inhibitor, an antibody or its antigen-binding fragment thereof, a peptide inhibitor, or a nucleotide-based inhibitor. In some embodiments, the IL15R inhibitor is a small molecule inhibitor, an antibody or its antigen-binding fragment thereof, a peptide inhibitor, or a nucleotide-based inhibitor. In some embodiments, the IL2R/IL15R inhibitor is a small molecule inhibitor, an antibody or its antigen-binding fragment thereof, a peptide inhibitor, or a nucleotide-based inhibitor. In some embodiments, the IL2R inhibitor is an IL2Rβ inhibitor. In some embodiments, the IL15R inhibitor is an IL15RB inhibitor. In some embodiments, the IL2R inhibitor is a CD122 inhibitor. In some embodiments, the IL15R inhibitor is a CD122 inhibitor. In some embodiments, the IL2R/IL15R inhibitor is a CD122 inhibitor. The IL2Rβ subunit and the IL15Rβ subunit are also known as CD122. In some embodiments, the antibody is an anti-CD122 antibody.

In some instances, patients with hematologic and lymphoid malignancies can benefit greatly from and often achieve long-term curative outcomes from allogeneic hematopoietic stem cell transplantation (alloHSCT) procedures. One of the main complications of alloHSCT is chronic graft versus host disease (cGvHD). Prophylaxis of cGvHD and successful treatment of cGvHD that do present in a subject are goals of medical practitioners hoping the minimize complications arising from alloHSCT. As pro-inflammatory mediators are thought to promote cGvHD, a balanced approach to prophylaxis or treatment in which pro-inflammatory molecules and signals are minimized while still enabling a desired therapeutic benefit of graft-versus-leukemia or graft-versus-tumor would be ideal.

### Combination Therapy for Treating GvHD (aGvHD and cGvHD)

Described herein are methods of prophylaxis for aGvHD and for cGvHD. Also described here are methods of treatment for aGvHD and for cGvHD. In one aspect, a method of prophylaxis for aGvHD comprises administering an effective amount of an IL15R inhibitor combined with a JAK inhibitor. In some cases, the IL15R inhibitor is an anti-CD122 antibody. In some embodiments, the administering inhibits IL2 and/or IL15 signaling. In some embodiments, the administering inhibits IL15 signaling. In some embodiments, the administering inhibits IL2 signaling and IL15 signaling. In some embodiments, the inhibition of IL2 and/or IL15 signaling occurs in cell types expressing intermediate affinity IL2R or IL15R composed of beta and gamma receptor subunits. In some embodiments, the administering an effective amount of an anti-CD122 antibody is combined with administering a JAK inhibitor. In some embodiments, the IL15R inhibitor disrupts or diminishes an IL15/IL15Rα complex from binding to an IL15Rβ/IL15Rγ complex. In some embodiments, the effective amount of the IL15R inhibitor does not significantly disrupt IL2 from binding to a high affinity IL2Rα/IL2Rβ/IL2Rγ complex. In some embodiments, the effective amount of the IL15R inhibitor does not significantly disrupt IL2 from signaling through a high affinity IL2Rα/IL2Rβ/IL2Rγ complex.

In one aspect, a method of prophylaxis for aGvHD comprises administering an effective amount of an IL2R inhibitor combined with a JAK inhibitor. In some cases, the IL2R inhibitor is an anti-CD122 antibody. In some embodiments, the administering inhibits IL2 and/or IL15 signaling. In some embodiments, the administering inhibits IL2 signaling. In some embodiments, the administering inhibits IL2 signaling and IL15 signaling. In some embodiments, the inhibition of IL2 and/or IL15 signaling occurs in cell types expressing intermediate affinity IL2R or IL15R composed of beta and gamma receptor subunits. In some embodiments, the administering an effective amount of an anti-CD122 antibody is combined with administering a JAK inhibitor. In some embodiments, the IL2R inhibitor disrupts or diminishes IL2 from binding to an IL2Rβ/IL2Rγ complex. In some embodiments, the effective amount of the IL2R inhibitor does not significantly disrupt IL2 from binding to a high affinity IL2Rα/IL2Rβ/IL2Rγ complex. In some embodiments, the effective amount of the IL2R inhibitor does not significantly disrupt IL2 from signaling through a high affinity IL2Rα/IL2Rβ/IL2Rγ complex.

In one aspect, a method of prophylaxis for aGvHD comprises administering an effective amount of an IL2R/IL15R inhibitor combined with a JAK inhibitor. In some embodiments, an IL2R/IL15R inhibitor is capable of inhibiting both IL2 signaling and IL15 signaling. In some cases, the IL2R/IL15R inhibitor is an anti-CD122 antibody. In some embodiments, the administering inhibits IL2 and/or IL15 signaling. In some embodiments, the administering inhibits IL2 signaling. In some embodiments, the administering inhibits IL15 signaling. In some embodiments, the administering inhibits IL2 signaling and IL15 signaling. In some embodiments, the inhibition of IL2 and/or IL15 signaling occurs in cell types expressing intermediate affinity IL2R or IL15R composed of beta and gamma receptor subunits. In some embodiments, the administering an effective amount of an anti-CD122 antibody is combined with administering a JAK inhibitor. In some embodiments, the IL2R/IL15R inhibitor disrupts or diminishes an IL15/IL15Rα complex from binding to an IL15Rβ/IL15Rγ complex. In some embodiments, the IL2R/IL15R inhibitor disrupts or diminishes IL2 from binding to an IL2Rβ/IL2Rγ complex. In some embodiments, the IL2R/IL15R inhibitor disrupts or diminishes both IL2 and IL15 from binding to an intermediate affinity IL-βγ receptor. In some embodiments, the effective amount of the IL2R/IL15R inhibitor does not significantly disrupt IL2 from binding to a high affinity IL2Rα/IL2Rβ/IL2Rγcomplex. In some embodiments, the effective amount of the IL2R/IL15R inhibitor does not significantly disrupt IL2 from signaling through a high affinity IL2Rα/IL2Rβ/IL2Rγ complex.

In another aspect, a method of prophylaxis for cGvHD comprises administering an effective amount of an IL15R inhibitor. In some cases, the IL15R inhibitor is an anti-CD122 antibody. In some embodiments, the method further comprises administering a JAK inhibitor. In some embodiments, the administering inhibits IL2 and/or IL15 signaling. In some embodiments, the administering inhibits IL15 signaling. In some embodiments, the inhibition of IL2 and/or IL15 signaling occurs in cell types expressing intermediate affinity IL2R or IL15R composed of beta and gamma receptor subunits. In some embodiments, the administering an effective amount of an anti-CD122 antibody is combined with administering a JAK inhibitor. In some embodiments, the IL15R inhibitor disrupts or diminishes an IL15/IL15Rα complex from binding to an IL15Rβ/IL15Rγ complex.

In another aspect, a method of prophylaxis for cGvHD comprises administering an effective amount of an IL2R inhibitor. In some cases, the IL2R inhibitor is an anti-CD122 antibody. In some embodiments, the method further comprises administering a JAK inhibitor. In some embodiments, the administering inhibits IL2 and/or IL15 signaling. In some embodiments, the administering inhibits IL2 signaling. In some embodiments, the inhibition of IL2 and/or IL15 signaling occurs in cell types expressing intermediate affinity IL2R or IL15R composed of beta and gamma receptor subunits. In some embodiments, the administering an effective amount of an anti-CD122 antibody is combined with administering a JAK inhibitor. In some embodiments, the IL2R inhibitor disrupts or diminishes IL2 from binding to an IL2Rβ/IL2Rγ complex.

In another aspect, a method of prophylaxis for cGvHD comprises administering an effective amount of an IL2R/IL15R inhibitor. In some cases, the IL2R/IL15R inhibitor is an anti-CD122 antibody. In some embodiments, the method further comprises administering a JAK inhibitor. In some embodiments, the administering inhibits IL2 and/or IL15 signaling. In some embodiments, the administering inhibits IL2 signaling. In some embodiments, the administering inhibits IL15 signaling. In some embodiments, the administering inhibits IL2 signaling and IL15 signaling. In some embodiments, the inhibition of IL2 and/or IL15 signaling occurs in cell types expressing intermediate affinity IL2R or IL15R composed of beta and gamma receptor subunits. In some embodiments, the administering an effective amount of an anti-CD122 antibody is combined with administering a JAK inhibitor. In some embodiments, the IL2R/IL15R inhibitor disrupts or diminishes an IL15/IL15Rα complex from binding to an IL15Rβ/IL15Rγ complex. In some embodiments, the IL2R/IL15R inhibitor disrupts or diminishes IL2 from binding to an IL2Rβ/IL2Rγ complex. In some embodiments, the IL2R/IL15R inhibitor disrupts or diminishes both IL2 and IL15 from binding to an intermediate affinity IL-βγ receptor.

In one aspect, a method for treatment of aGvHD comprises administering a therapeutically effective amount of an IL15R inhibitor combined with a JAK inhibitor. In some embodiments, the IL15R inhibitor is an anti-CD122 antibody. In some embodiments, the administering inhibits IL2 and/or IL15 signaling. In some embodiments, the administering inhibits IL15 signaling. In some embodiments, the inhibition of IL2 and/or IL15 signaling occurs in cell types expressing intermediate affinity IL2R or IL15R composed of beta and gamma receptor subunits. In some embodiments, the administering a therapeutically effective amount of an anti-CD122 antibody is combined with administering a JAK inhibitor. In some embodiments, the IL15R inhibitor disrupts or diminishes an IL15/IL15Rα complex from binding to an IL15Rβ/IL15Rγ complex.

In one aspect, a method for treatment of aGvHD comprises administering a therapeutically effective amount of an IL2R inhibitor combined with a JAK inhibitor. In some embodiments, the IL2R inhibitor is an anti-CD122 antibody. In some embodiments, the administering inhibits IL2 and/or IL15 signaling. In some embodiments, the administering inhibits IL2 signaling. In some embodiments, the inhibition of IL2 and/or IL15 signaling occurs in cell types expressing intermediate affinity IL2R or IL15R composed of beta and gamma receptor subunits. In some embodiments, the administering a therapeutically effective amount of an anti-CD122 antibody is combined with administering a JAK inhibitor.

In one aspect, a method for treatment of aGvHD comprises administering a therapeutically effective amount of an IL2R/IL15R inhibitor combined with a JAK inhibitor. In some embodiments, the IL2R/IL15R inhibitor is an anti-CD122 antibody. In some embodiments, the administering inhibits IL2 and/or IL15 signaling. In some embodiments, the administering inhibits IL2 signaling. In some embodiments, the administering inhibits IL15 signaling. In some embodiments, the administering inhibits IL2 signaling and IL15 signaling. In some embodiments, the inhibition of IL2 and/or IL15 signaling occurs in cell types expressing intermediate affinity IL2R or IL15R composed of beta and gamma receptor subunits. In some embodiments, the administering a therapeutically effective amount of an anti-CD122 antibody is combined with administering a JAK inhibitor. In some embodiments, the IL2R/IL15R inhibitor disrupts or diminishes an IL15/IL15Rα complex from binding to an IL15Rβ/IL15Rγ complex.

In one aspect, a method for treatment of cGvHD comprises administering a therapeutically effective amount of an IL15R inhibitor. In some embodiments, the IL15R inhibitor is an anti-CD122 antibody. In some embodiments, the method further comprises administering a JAK inhibitor. In some embodiments, the administering inhibits IL2 and/or IL15 signaling. In some embodiments, the administering inhibits IL15 signaling. In some embodiments, the inhibition of IL2 and/or IL15 signaling occurs in cell types expressing intermediate affinity IL2R or IL15R composed of beta and gamma receptor subunits. In some embodiments, the administering a therapeutically effective amount of an anti-CD122 antibody is combined with administering a JAK inhibitor.

In one aspect, a method for treatment of cGvHD comprises administering a therapeutically effective amount of an IL2R inhibitor. In some embodiments, the IL2R inhibitor is an anti-CD122 antibody. In some embodiments, the method further comprises administering a JAK inhibitor. In some embodiments, the administering inhibits IL2 and/or IL15 signaling. In some embodiments, the administering inhibits IL2 signaling. In some embodiments, the inhibition of IL2 and/or IL15 signaling occurs in cell types expressing intermediate affinity IL2R or IL15R composed of beta and gamma receptor subunits. In some embodiments, the administering a therapeutically effective amount of an anti-CD122 antibody is combined with administering a JAK inhibitor.

In one aspect, a method for treatment of cGvHD comprises administering a therapeutically effective amount of an IL2R/IL15R inhibitor. In some embodiments, the IL2R/IL15R inhibitor is an anti-CD122 antibody. In some embodiments, the method further comprises administering a JAK inhibitor. In some embodiments, the administering inhibits IL2 and/or IL15 signaling. In some embodiments, the administering inhibits IL2 signaling. In some embodiments, the administering inhibits IL15 signaling. In some embodiments, the administering inhibits IL2 signaling and IL15 signaling. In some embodiments, the inhibition of IL2 and/or IL15 signaling occurs in cell types expressing intermediate affinity IL2R or IL15R composed of beta and gamma receptor subunits. In some embodiments, the administering a therapeutically effective amount of an anti-CD 122 antibody is combined with administering a JAK inhibitor.

### IL15 Receptor Inhibitors

Provided herein are methods of treating cGvHD by administering an effective amount of an IL15R inhibitor. Also provided herein are methods of treating aGvHD by administering an effective amount of an IL15R inhibitor in combination with a JAK inhibitor. Various IL15R inhibitors are contemplated for use in methods provided herein. In some cases, the IL15R inhibitor comprises an antibody or antigen-binding fragment thereof. In some cases, the IL15R inhibitor comprises an inhibitory peptide. In some cases, the IL15R inhibitor comprises an inhibitory nucleic acid. In some embodiments, the IL15R inhibitor comprises a small molecule.

In some cases, the IL15R inhibitor comprises an antibody or antigen-binding fragment thereof. In some embodiments, the antibody comprises an Immunoglobulin G (IgG) antibody or variant thereof. In some embodiments, the IgG antibody or variant thereof comprises an IgG1antibody or variant thereof. In some embodiments, the IgG antibody or variant thereof comprises an IgG2 antibody or variant thereof. In some embodiments, the IgG antibody or variant thereof comprises an IgG3 antibody or variant thereof. In some embodiments, the IgG antibody or variant thereof comprises an IgG4 antibody or variant thereof. In some embodiments, the antibody comprises an IgA antibody. In some embodiments, the antibody comprises an IgM antibody. In some embodiments, the antibody comprises an IgE antibody. In some embodiments, the antibody or antigen-binding fragment thereof comprises an IgG-scFv. In some embodiments, the antibody or antigen-binding fragment thereof comprises a nanobody. In some embodiments, the antibody or antigen-binding fragment thereof comprises a mini-antibody. In some embodiments, the antibody or antigen-binding fragment thereof comprises a scFv-CH3 KIH. In some embodiments, the antibody or antigen-binding fragment thereof comprises a Fab-scFv-Fc KIH. In some embodiments, the antibody or antigen-binding fragment thereof comprises a Fab-scFv. In some embodiments, the antibody or antigen-binding fragment thereof comprises a scFv-CH-CL-scFv. In some embodiments, the antibody or antigen-binding fragment thereof comprises a Fab′. In some embodiments, the antibody or antigen-binding fragment thereof comprises a Fab′, a F(ab′)2, a F(ab′)3, a F(ab′)2-scFv2, an scFv, an scFv-KIH, or a Fab-scFv-Fc. In some embodiments, the antibody or antigen-binding fragment thereof comprises an intrabody. In some embodiments, the IL15R antibody comprises an IL15Rβ antibody. In some exemplary embodiments, the IL15Rβ antibody is a rat anti-mouse IL15Rβ antibody (clone TM-β1), a rat-mouse chimeric anti-mouse IL15Rβ antibody (ChMBC7), a mouse anti-human IL15Rβ antibody (MIKB1), a mouse anti-human IL15Rβ monoclonal antibody (TU27), a mouse anti-human IL15Rβ monoclonal antibody, a humanized mouse anti-human IL15Rβ monoclonal antibody, a rat anti-mouse IL15Rβ IgG2a monoclonal antibody (5H4), a mouse anti-human IL15Rβ IgG1 monoclonal antibody (clone 27302, ThermoFisher), a mouse anti-human IL15Rβ monoclonal antibody (A41), a rabbit anti-human IL15Rβ polyclonal antibody, a sheep anti-human IL15Rβ polyclonal antibody, or a rabbit anti-phospho IL15Rβ polyclonal antibody. In some embodiments, the IL15R antibody comprises an IL15Rα antibody. In some cases, the IL15Rα antibody comprises an anti-IL15Rα antibody, mouse anti-human IL-5Rα monoclonal antibody (eBioJM7A4), a mouse anti-human IL15Rα monoclonal antibody (M165), a mouse anti-human IL15Rα IgG2a monoclonal antibody, a rabbit anti-human IL15Rα polyclonal antibody, a goat anti-human IL15Rα polyclonal antibody, a mouse anti-human IL15Rα monoclonal antibody (OTI3D5), or a mouse anti-human IL15Rα monoclonal antibody (OTI7F4). In some cases, the IL15R antibody comprise an IL15Rγ antibody. In some embodiments, the IL15Rγ antibody is an anti-mouse CD132 antibody, a rat anti-human CD132 monoclonal antibody (TUGh4), a rat anti-mouse CD132 monoclonal antibody (TUGm2), a goat anti-human CD132 polyclonal antibody (AF284), a rabbit anti-human CD132 polyclonal antibody (sc271-60), a rabbit anti-mouse CD132 recombinant monoclonal antibody (11), or a mouse anti-human CD132 IgG2b monoclonal antibody (clone 633162, ThermoFisher).

In some cases, the IL15R inhibitor comprises an inhibitory peptide. In some embodiments, the peptide inhibitor comprises a soluble IL15Rα, an IL15 antagonist isoform (lacking exon 6), a synthetic peptide, an IL15:Fc mutant fusion, an IL15 mutant/Fcγ2a fusion protein, an IL15 mutant polypeptide, an IL2 mutein, and IL15.IL15Rα fusion molecule, a peptide that binds IL15Rα and inhibits IL15, or a peptide inhibitor of IL2, IL9, or IL15.

In some cases, the IL15R inhibitor is a small molecule. In some embodiments, the small molecule comprises Cefazolin, Ro26-4550, SP4206, Abt-737, Nutlin-2, a benzoic acid derivative, an amine derivative, compound 3, 8-bromo-N-(2.4-dichlorophenyl) octanamide, or 8-bromo-N-(2.4-difluorophenyl) octanamide.

In some cases, the IL15R inhibitor is an inhibitory nucleic acid. In some embodiments, the inhibitory nucleic acid is an antisense RNA, a siRNA, a shRNA, an antisense oligonucleotide, a morpholino oligonucleotide, or an RNAi molecule comprising a LNA or a PNA that specifically inhibits expression of an IL15R mRNA.

In some embodiments, the IL15R inhibitor is an antibody that binds to CD122. In some embodiments, the IL15R inhibitor is a CD122 inhibitor. In some embodiments, the IL15R inhibitor is an IL15Rβ inhibitor. In some instances, the antibodies that bind to CD122 are monoclonal antibodies. In certain aspects, disclosed herein is an anti-CD122 antibody. In some instances, the anti-CD122 antibody specifically binds to mammalian CD122. In some instances. the anti-CD122 antibody specifically binds to a rodent (e.g., mouse or rat) CD122. In some instances, the anti-CD122 antibody specifically binds to a murine (e.g., mouse or related species) CD122. In some instances, the anti-CD122 antibody specifically binds to a human CD122. In some instances, the anti-CD122 antibody specifically binds to an extracellular portion of CD122. In some instances, the anti-CD122 antibody specifically binds to an extracellular portion of rodent (e.g., mouse or rat) CD122. In some instances, the anti-CD122 antibody specifically binds to an extracellular portion of murine (e.g., mouse or related species) CD122. In some instances, the anti-CD122 antibody specifically binds to an extracellular portion of human CD122. In some instances, the anti-CD122 antibody is made of chimeric amino acid sequences some of which are murine-derived and some of which are human-derived. In some instances, the anti-CD122 antibody is made with complementarity-determining regions (CDRs) that have been incorporated into an antibody scaffold. In some instances, the anti-CD122 antibody is made with complementarity-determining regions (CDRs) incorporated into a human antibody variable region framework. In some instances, the human antibody variable region framework has been sequence-optimized to retain CD122 affinity with the engrafted mouse CDR sequences. In some instances, the anti-CD122 antibody is a humanized antibody. In some instances, the anti-CD122 antibody is a humanized anti-human CD122 antibody designated Antibody 1. In some instances, the anti-CD122 antibody is a humanized anti-human CD122 antibody designated Antibody 2. In some embodiments, the anti-CD122 antibody is a rat anti-mouse CD122 antibody designated Antibody 3. In some instances, the anti-CD122 antibody is a mouse anti-human CD122 antibody designated Antibody 4. Antibody 4 is derived from Clone TU27, obtained from BioLegend®, Catalog #339015. In some instances, the anti-CD122 antibody is a fully human antibody. In some instances, the anti-CD122 antibody is a chimeric antibody.

In aspects described herein, an IL15R inhibitor can function as an IL2R inhibitor and an IL15R inhibitor. In some embodiments, this function as an IL2R inhibitor and an IL15R inhibitor is referred to as an IL2R/IL15R inhibitor. Intermediate and high affinity IL2 and IL15 receptors share several polypeptide components, namely the beta receptor subunit (CD122) and the gamma receptor subunit (CD132). In some instances, the intermediate affinity IL-βγ receptor, composed of CD122 and CD132 subunits, can bind to IL2 ligand and IL15 ligand. In some embodiments, the IL2R/IL15R inhibitor inhibits binding of both IL2 ligand and IL15 ligand to the intermediate affinity IL-βγ receptor. In some embodiments, the IL2R/IL15R inhibitor inhibits binding of both IL2 ligand and IL15 ligand to the CD122 component of the intermediate affinity IL-βγ receptor. In some embodiments, the IL2R/IL15R inhibitor inhibits binding of both IL2 ligand and IL15 ligand to the CD132 component of the intermediate affinity IL-βγ receptor. In some embodiments, an IL2R/IL15R inhibitor may bind competitively to one or more components of the intermediate affinity IL-βγ receptor compared to IL2 ligand. IL15 ligand, or IL2 ligand and IL15 ligand. In some embodiments, an IL2R/IL15R inhibitor may bind competitively to the CD122 subunit of the intermediate affinity IL-βγ receptor compared to IL2 ligand. IL15 ligand, or IL2 ligand and IL15 ligand. In some embodiments, an IL2R/IL15R inhibitor may bind competitively to the CD132 subunit of the intermediate affinity IL-βγ receptor compared to IL2 ligand. IL15 ligand, or IL2 ligand and IL15 ligand. In some embodiments, the competitive binding of the IL2R/IL15R inhibitor prevents both IL2 ligand and IL15 ligand from binding to the intermediate affinity IL-βγ receptor expressed in target cells and prevents stimulation of IL2-mediated signal transduction and IL15-mediated signal transduction. In some embodiments, the competitive binding of the IL2R/IL15R inhibitor prevents both IL2ligand and IL15 ligand from binding to the intermediate affinity IL-βγ receptor expressed in target cells and prevents stimulation of IL2-mediated signal transduction and IL15-mediated signal transduction mediated through the intermediate affinity IL-βγ receptor. In some embodiments, the intermediate affinity IL-βγ receptor is expressed in target cells. In some embodiments, the target cells expressing the intermediate affinity IL-βγ receptor are T cells. In some embodiments, the target cells expressing the intermediate affinity IL-βγ receptor are NK cells. In some embodiments, the target cells expressing the intermediate affinity IL-βγ receptor are T cells and NK cells. In some embodiments, methods described herein comprising administering an effective amount of an IL2R inhibitor, an IL15R inhibitor, or an IL2R/IL15R inhibitor to a subject results in inhibition of IL2 binding. IL15 binding, or IL2 binding and IL15 binding to the intermediate affinity IL-βγ receptor and reduces activation of T cells, NK cells, or both T cells and NK cells. In some embodiments, methods described herein comprising administering an effective amount of an IL2R inhibitor, an IL15R inhibitor, or an IL2R/IL15R inhibitor to a subject results in inhibition of IL2 binding. IL15 binding, or IL2 binding and IL15 binding to the intermediate affinity IL-βγ receptor and prevents activation of T cells, NK cells, or both T cells and NK cells. In some embodiments, methods described herein comprising administering an effective amount of an IL2R inhibitor, an IL15R inhibitor, or an IL2R/IL15R inhibitor to a subject results in inhibition of IL2 binding. IL15 binding, or IL2 binding and IL15 binding to the intermediate affinity IL-βγ receptor and reduces proliferation of T cells, NK cells, or both T cells and NK cells. In some embodiments, methods described herein comprising administering an effective amount of an IL2R inhibitor, an IL15R inhibitor, or an IL2R/IL15R inhibitor to a subject results in inhibition of IL2 binding. IL15 binding, or IL2 binding and IL15 binding to the intermediate affinity IL-βγ receptor and prevents proliferation of T cells. NK cells, or both T cells and NK cells. In some embodiments, the IL2R/IL15R inhibitor does not significantly compete with IL2 and/or IL15 ligand binding with a high affinity IL-αβγ receptor. In some embodiments, the IL2R/IL15R inhibitor that binds to CD122 does not significantly compete with IL2 and/or IL15 ligand binding with a high affinity IL-αβγ receptor. In some embodiments, the IL2R/IL15R inhibitor that binds to CD122 does not significantly compete with IL2 and/or IL15 ligand binding with a high affinity IL-αβγ receptor expressed in one or more non-target cell. In some embodiments, one or more non-target cells comprise regulatory T cells (Tregs). In some instances, Tregs express the high affinity ILαβγ receptor. In some embodiments, methods described herein comprising administering an effective amount of an IL2R inhibitor, an IL15R inhibitor, or an IL2R/IL15R inhibitor to a subject do not significantly interfere with IL2 ligand binding to the high affinity IL-αβγ receptor expressed in non-target cells. In some embodiments, methods described herein comprising administering an effective amount of an IL2R inhibitor, an IL15R inhibitor, or an IL2R/IL15R inhibitor to a subject do not significantly interfere with IL2 ligand binding to the high affinity IL-αβγ receptor expressed in Tregs. In some embodiments, activation and/or proliferation of Tregs is not inhibited. In some embodiments, an IL2R inhibitor can function as an IL2R/IL15R inhibitor.

### IL2 Receptor Inhibitors

Provided herein are methods of treating cGvHD by administering an effective amount of an IL2R inhibitor. Also provided herein are methods of treating aGvHD by administering an effective amount of an IL2R inhibitor in combination with a JAK inhibitor. Various IL2R inhibitors are contemplated for use in methods provided herein. In some cases, the IL2R inhibitor comprises an antibody or antigen-binding fragment thereof. In some cases, the IL2R inhibitor comprises an inhibitory peptide. In some cases, the IL2R inhibitor comprises an inhibitory nucleic acid. In some embodiments, the IL2R inhibitor comprises a small molecule. In some embodiments, an IL2R inhibitor can function as an IL2R/IL15R inhibitor.

In some cases, the IL2R inhibitor comprises an antibody or antigen-binding fragment thereof. In some embodiments, the antibody comprises an Immunoglobulin G (IgG) antibody or variant thereof. In some embodiments, the IgG antibody or variant thereof comprises an IgG1 antibody or variant thereof. In some embodiments, the IgG antibody or variant thereof comprises an IgG2 antibody or variant thereof. In some embodiments, the IgG antibody or variant thereof comprises an IgG3 antibody or variant thereof. In some embodiments, the IgG antibody or variant thereof comprises an IgG4 antibody or variant thereof. In some embodiments, the antibody comprises an IgA antibody. In some embodiments, the antibody comprises an IgM antibody. In some embodiments, the antibody comprises an IgE antibody. In some embodiments, the antibody or antigen-binding fragment thereof comprises an IgG-scFv. In some embodiments, the antibody or antigen-binding fragment thereof comprises a nanobody. In some embodiments, the antibody or antigen-binding fragment thereof comprises a mini-antibody. In some embodiments, the antibody or antigen-binding fragment thereof comprises a scFv-CH3 KIH. In some embodiments, the antibody or antigen-binding fragment thereof comprises a Fab-scFv-Fc KIH. In some embodiments, the antibody or antigen-binding fragment thereof comprises a Fab-scFv. In some embodiments, the antibody or antigen-binding fragment thereof comprises a scFv-CH-CL-scFv. In some embodiments, the antibody or antigen-binding fragment thereof comprises a Fab′. In some embodiments, the antibody or antigen-binding fragment thereof comprises a Fab′, a F(ab′)2, a F(ab′)3, a F(ab′)2-scFv2, an scFv, an scFy-KIH, or a Fab-scFv-Fc. In some embodiments, the antibody or antigen-binding fragment thereof comprises an intrabody. In some embodiments, the IL2R antibody comprises an IL2Rβ antibody. In some exemplary embodiments, the IL2Rβ antibody is a rat anti-mouse IL2Rβ antibody (clone TM-β1), a rat-mouse chimeric anti-mouse IL2Rβ antibody (ChMBC7), a mouse anti-human IL2Rβ antibody (MIKB1), a mouse anti-human IL2Rβ monoclonal antibody (TU27), a mouse anti-human IL2Rβ monoclonal antibody, a humanized mouse anti-human IL2Rβ monoclonal antibody, a rat anti-mouse IL2Rβ IgG2a monoclonal antibody (5H4), a mouse anti-human IL2Rβ IgGI monoclonal antibody (clone 27302, ThermoFisher), a mouse anti-human IL2Rβ monoclonal antibody (A41), a rabbit anti-human IL2Rβ polyclonal antibody, a sheep anti-human IL2Rβ polyclonal antibody, or a rabbit anti-phospho IL2Rβ polyclonal antibody. In some embodiments, the IL2R antibody comprises an IL2Rα antibody. In some cases, the IL2Rα antibody is an anti-IL15Rα antibody, a mouse anti-human IgG1 monoclonal antibody (Clone BC96, ThermoFisher Catalog #12-0259-80), or a mouse anti-human IgG1 monoclonal antibody (Clone CD25-4E3, ThermoFisher Catalog #17-0257-42). In some cases, the IL2R antibody comprise an IL2Rγ antibody. In some embodiments, the IL2Rγ antibody is an anti-mouse CD132 antibody, a rat anti-human CD132 monoclonal antibody (TUGh4), a rat anti-mouse CD132 monoclonal antibody (TUGm2), a goat anti-human CD132 polyclonal antibody (AF284), a rabbit anti-human CD132 polyclonal antibody (sc271-60), a rabbit anti-mouse CD132 recombinant monoclonal antibody (11), or a mouse anti-human CD132 IgG2b monoclonal antibody (clone 633162, ThermoFisher).

In some cases, the IL2R inhibitor comprises an inhibitory peptide. In some embodiments, the peptide inhibitor comprises a soluble IL2Rα, an IL2 antagonist isoform, a synthetic peptide, an IL2:Fc mutant fusion, an IL2 mutant polypeptide, an IL2 mutein, a peptide that binds IL2Rα and inhibits IL2, or a peptide inhibitor of IL2, IL9, or IL15.

In some cases, the IL2R inhibitor is a small molecule. In some embodiments, the small molecule is an acylphenylalanine derivative that mimics the R38-F42 region of IL2, Ro26-4550, or SP4206.

In some cases, the IL2R inhibitor is an inhibitory nucleic acid. In some embodiments. the inhibitory nucleic acid is an antisense RNA, a siRNA, a shRNA, an antisense oligonucleotide, a morpholino oligonucleotide, or an RNAi molecule comprising a LNA or a PNA that specifically inhibits expression of an IL15R mRNA.

In some embodiments, the IL2R inhibitor is an antibody that binds to CD122. In some embodiments, the IL2R inhibitor is a CD122 inhibitor. In some embodiments, the IL2R inhibitor is an IL2Rβ inhibitor. In some embodiments, the CD122 antibody functions as an inhibitor of IL2 signaling and IL15 signaling. In some instances, the antibodies that bind to CD122 are monoclonal antibodies. In certain aspects, disclosed herein is an anti-CD122 antibody. In some instances, the anti-CD122 antibody specifically binds to mammalian CD122. In some instances, the anti-CD122 antibody specifically binds to a rodent (e.g., mouse or rat) CD122. In some instances, the anti-CD122 antibody specifically binds to a murine (e.g., mouse or related species) CD122. In some instances, the anti-CD122 antibody specifically binds to a human CD122. In some instances, the anti-CD122 antibody specifically binds to an extracellular portion of CD122. In some instances, the anti-CD122 antibody specifically binds to an extracellular portion of rodent (e.g., mouse or rat) CD122. In some instances, the anti-CD122 antibody specifically binds to an extracellular portion of murine (e.g., mouse or related species) CD122. In some instances, the anti-CD122 antibody specifically binds to an extracellular portion of human CD122. In some instances, the anti-CD122 antibody is made of chimeric amino acid sequences some of which are murine-derived and some of which are human-derived.

In some instances, the anti-CD122 antibody is made with complementarity-determining regions (CDRs) that have been incorporated into an antibody scaffold. In some instances, the anti-CD122 antibody is made with complementarity-determining regions (CDRs) incorporated into a human antibody variable region framework. In some instances, the human antibody variable region framework has been sequence-optimized to retain CD122 affinity with the engrafted mouse CDR sequences. In some instances, the anti-CD122 antibody is a humanized antibody. In some instances, the anti-CD122 antibody is a humanized anti-human CD122 antibody designated Antibody 1. In some instances, the anti-CD122 antibody is a humanized anti-human CD122 antibody designated Antibody 2. In some embodiments, the anti-CD122 antibody is a rat anti-mouse CD122 antibody designated Antibody 3. In some instances, the anti-CD122 antibody is a mouse anti-human CD122 antibody designated Antibody 4. Antibody 4 is derived from Clone TU27, obtained from BioLegend®, Catalog #339015. In some instances, the anti-CD122 antibody is a fully human antibody. In some instances, the anti-CD122 antibody is a chimeric antibody.

### JAK inhibitors

In some instances. JAK inhibitors include JAK1 inhibitor, JAK2 inhibitor, and/or JAK3 inhibitor. In some instances. JAK inhibitors include JAK1/3 inhibitor, JAK1/2 inhibitor, or JAK1/2/3 inhibitor. In some instances, JAK inhibitor is a small molecule inhibitor, an antibody or antigen-fragment thereof, or a peptide inhibitor. In some embodiments, the JAK inhibitor administered to the subject is selected from abrocitinib, baricitinib, delgocitinib, fedratinib, filgotinib, oclacitinib, pacritinib, peficitinib, ruxolitinib, tofacitinib, itacitinib, or upadacitinib. In some embodiments, the JAK inhibitor administered to the subject comprises ruxolitinib. Ruxolitinib can effectively target JAK1 and JAK2 for inhibition of JAK-mediated signaling. In some embodiments, the JAK inhibitor administered to the subject consists essentially of ruxolitinib. In some embodiments, the ruxolitinib is administered orally.

### Production and Manufacture of Antibodies or Antigen-Binding Fragments Thereof

In some embodiments, polypeptides described herein (e.g., antibodies or antigen-binding fragments thereof) are produced using any method known in the art to be useful for the synthesis of polypeptides (e.g., antibodies), in particular, by chemical synthesis or by recombinant expression, and are preferably produced by use of a recombinant expression technique.

In some instances, an antibody or antigen-binding fragment thereof is expressed recombinantly, and the nucleic acid encoding the antibody or its antigen-binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 5′ and 3′ ends of the sequence or by cloning using an oligonucleotide specific for the particular nucleic acid sequence.

In some instances, an antibody or its antigen-binding fragment thereof is optionally made by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937.

In some embodiments, techniques developed for the production of “chimeric antibodies” (Morrison et al., 1984, Proc. Natl. Acad. Sci. 81:851-855: Neuberger et al., 1984. Nature 312:604-608: Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region, e.g., humanized antibodies.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, a nucleic acid sequence encodes the antibodies disclosed herein. In some embodiments, the polynucleotide sequence encoding the antibodies is operatively coupled to a eukaryotic regulatory sequence. In some embodiments, a cell comprises the nucleic acid sequence. In some embodiments, a cell comprises a nucleic acid encoding the antibodies disclosed herein. In some embodiments, the cell comprises a prokaryotic cell. In some embodiments, the prokaryotic cell is an Escherichia coli cell. In some embodiments, the cell comprises a eukaryotic cell. In some embodiments, the eukaryotic cell is a Chinese Hamster Ovary (CHO) cell, a HEK293 cell, a BHK cell, an NSO murine myeloma cell, or a PER.C6® human cell. In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific aspects, the expression of the antibody is regulated by a constitutive, an inducible or a tissue-specific promoter. Standard cell lines and methods for the production of antibodies from a large-scale cell culture are known in the art. See e.g., Li et al., “Cell culture processes for monoclonal antibody production.” Mabs. 2010 September-Oct: 2(5): 466-477.

In certain aspects, described herein is a method of making antibodies comprising culturing a cell comprising a nucleic acid encoding an antibody under conditions in vitro sufficient to allow production and secretion of the antibody. In some embodiments, antibodies are harvested from the cell culture medium. In some cases, the harvesting further comprises one or more purification steps to remove live cells, cellular debris, non-antibody proteins or polypeptides, undesired salts, buffers, and medium components. In certain aspects, the additional purification step(s) include centrifugation, ultracentrifugation, protein A, protein G. protein A/G, or protein L purification, and/or ion exchange chromatography.

### Pharmaceutical Compositions

Provided here are pharmaceutical compositions comprising an IL15R inhibitor and at least one pharmaceutically acceptable carrier, excipient, or diluent. Also provided here are pharmaceutical compositions comprising an IL2R inhibitor and at least one pharmaceutically acceptable carrier, excipient, or diluent. In some embodiments, the composition further comprises a JAK inhibitor. In some embodiments, the IL15R inhibitor or the IL2R inhibitor is an antibody or an antigen-binding fragment thereof. In some embodiments, the antibody or antigen-binding fragment thereof binds to IL15Rα (CD215), IL15Rβ (CD122), or IL15Rγ (CD132). In some embodiments, the antibody or antigen-binding fragment thereof binds to IL2Rα (CD25), IL2Rβ (CD122), or IL2Rγ (CD132). In some embodiments, the antibody or antigen-binding fragment thereof is an anti-CD122 antibody or anti-CD122 antigen-binding fragment thereof. In some embodiments, the pharmaceutical composition comprises an anti-CD122 antibody described herein. In some embodiments, the antibody or antigen-binding fragment thereof binds to human CD122. In some embodiments, the antibody or an antigen-binding fragment thereof and at least one pharmaceutically acceptable carrier is formulated into a pharmaceutical formulation. In some embodiments, the pharmaceutical formulation is chosen based on a preferred route of administration of the antibody or antigen-binding fragment thereof to a subject.

In some embodiments, the pharmaceutical formulations include, but are not limited to. aqueous liquid dispersions, self-emulsifying dispersions, solid solutions, liposomal dispersions, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation includes multiparticulate formulations. In some instances, the pharmaceutical formulation includes nanoparticle formulations. In some instances, nanoparticles comprise cMAP, cyclodextrin, or lipids. In some cases, nanoparticles comprise solid lipid nanoparticles, polymeric nanoparticles, self-emulsifying nanoparticles, liposomes, microemulsions, or micellar solutions. Additional exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanorods, nanoropes, and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, bismuth, magnesium, calcium, strontium, barium, lithium, sodium, potassium, boron, silicon, phosphorus, germanium, arsenic, antimony, and combinations, alloys or oxides thereof.

In some instances, a nanoparticle includes a core or a core and a shell, as in a core-shell nanoparticle. In some instances, nanoparticles comprise nanospheres or nanocapsules.

In some instances, a nanoparticle is further coated with molecules for attachment of functional elements (e.g., with one or more of a polynucleic acid molecule or binding moiety described herein). In some instances, a coating comprises chondroitin sulfate, dextran sulfate, carboxymethyl dextran, alginate, pectin, carrageenan, fucoidan, agaropectin, porphyran, karaya gum, xanthan gum, hyaluronic acids, glucosamine, galactosamine, chitosan, polyglutamic acid, polyaspartic acid, lysozyme, cytochrome C, trypsinogen, chymotrypsin, α-chymotrypsin, polylysine, polyarginine, histone, protamine, ovalbumin, dextrin, or cyclodextrin.

In some embodiments, the pharmaceutical formulations described herein are administered to a subject by multiple administration routes, including but not limited to, parenteral (e.g., intravenous, subcutaneous, intramuscular), oral, intranasal, buccal, vaginal, rectal, or transdermal administration routes. In some instances, the pharmaceutical composition describe herein is formulated for parenteral (e.g., intravenous, subcutaneous, intramuscular, intra-arterial, intraperitoneal, intrathecal, intracerebral, intracerebroventricular, or intracranial) administration. In other instances, the pharmaceutical composition describe herein is formulated for oral administration. In still other instances, the pharmaceutical composition describe herein is formulated for intranasal administration.

### Pharmaceutically Acceptable Excipients, Carriers, And Diluents

Compositions comprising the IL15R inhibitor and optionally the JAK inhibitor of the current disclosure are included in a pharmaceutical composition comprising one or more pharmaceutically acceptable excipients, carriers, and diluents. In some embodiments, the antibodies of the current disclosure are administered suspended in a sterile solution. In some embodiments, the antibodies of the current disclosure are administered suspended in an isotonic solution. In some instances, the pharmaceutical formulation includes one or more salts in an amount required to bring osmolality of the composition into an acceptable range. Such salts include those having sodium, potassium or ammonium cations and chloride, citrate, ascorbate. borate, phosphate, bicarbonate, sulfate, thiosulfate or bisulfite anions: suitable salts include sodium chloride, potassium chloride, sodium thiosulfate, sodium bisulfite and ammonium sulfate. In certain aspects, the solution comprises about 0.9% NaCl. In certain aspects, the solution comprises about 5.0% dextrose. In certain aspects, the solution further comprises one or more of: buffers, for example, acetate, citrate, histidine, succinate, phosphate, bicarbonate and Tris(hydroxymethyl)aminomethane; surfactants, for example, polysorbate 80 (Tween 80), polysorbate 20 (Tween 20): polyol/disaccharide/polysaccharides, for example, glucose, dextrose, mannose, mannitol, sorbitol, sucrose, and dextran 40; amino acids, for example, glycine or arginine: antioxidants, for example, ascorbic acid, methionine: or chelating agents, for example. EDTA or EGTA. In certain aspects, the pharmaceutical composition comprises a biodegradable or bioabsorbable carrier such as polylactic acid (PLA), polyglycolic acid (PGA). or Poly(D,L-lactic-coglycolic-acid) (PLGA). %

In certain aspects, the antibodies of the current disclosure are shipped and/or stored lyophilized and can then be reconstituted before administration. In certain aspects, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated. whether reconstituted or not, can be buffered at a certain pH, generally less than about 7.5. In certain aspects, the pH can be between 4.5 and 7.5. 4.5 and 7.0. 4.5 and 6.5, 4.5 and 6.0, or 5.5 or 5.0.

### Delivery of IL15R Inhibitors or IL2R Inhibitors and JAK Inhibitors

In some aspects, an IL15R inhibitor is delivered to the subject by a certain route of administration. In some embodiments, the IL15R inhibitor is delivered systemically, locally, intradermally, subcutaneously, or topically. In some embodiments, the IL15R inhibitor delivered systemically is administered by intravenous injection, by subcutaneous injection, by enteral administration, or through inhalation. In some embodiments, the IL15R inhibitor delivered systemically is administered by intravenous injection according to a treatment regimen specifying a frequency of administration. In some embodiments, the IL15R inhibitor delivered by enteral administration is administered orally. In some embodiments, the IL15R inhibitor delivered by enteral administration is administered vaginally or rectally. In some embodiments, the IL15R inhibitor delivered locally is administered topically. In some embodiments, the IL15R inhibitor delivered locally is administered subcutaneously. In some embodiments, the IL15R inhibitor delivered locally is administered intradermally.

In some aspects, an IL2R inhibitor is delivered to the subject by a certain route of administration. In some embodiments, the IL2R inhibitor is delivered systemically, locally, intradermally, subcutaneously, or topically. In some embodiments, the IL2R inhibitor delivered systemically is administered by intravenous injection, by subcutaneous injection, by enteral administration, or through inhalation. In some embodiments, the IL2R inhibitor delivered systemically is administered by intravenous injection according to a treatment regimen specifying a frequency of administration. In some embodiments, the IL2R inhibitor delivered by enteral administration is administered orally. In some embodiments, the IL2R inhibitor delivered by enteral administration is administered vaginally or rectally. In some embodiments, the IL2R inhibitor delivered locally is administered topically. In some embodiments, the IL2R inhibitor delivered locally is administered subcutaneously. In some embodiments, the IL2R inhibitor delivered locally is administered intradermally.

In some aspects, a JAK inhibitor is delivered to the subject by a certain route of administration. In some embodiments, the JAK inhibitor is delivered systemically, locally, intradermally, subcutaneously, or topically. In some embodiments, the JAK inhibitor delivered systemically is administered by intravenous injection, by subcutaneous injection, by enteral administration, or through inhalation. In some embodiments, the JAK inhibitor delivered systemically is administered by intravenous injection according to a treatment regimen specifying a frequency of administration. In some embodiments, the JAK inhibitor delivered by enteral administration is administered orally. In some embodiments, the JAK inhibitor delivered systemically by oral administration is administered according to a treatment regimen specifying a frequency of administration. In some embodiments, the JAK inhibitor delivered by enteral administration is administered vaginally or rectally. In some embodiments, the JAK inhibitor delivered locally is administered topically. In some embodiments, the JAK inhibitor delivered locally is administered subcutaneously. In some embodiments, the JAK inhibitor delivered locally is administered intradermally.

In some aspects, an IL15R inhibitor and a JAK inhibitor are administered as a combination therapy. In some embodiments, the IL15R inhibitor and the JAK inhibitor are co-administered. In some instances, the IL15R inhibitor and the JAK inhibitor are formulated in a single pharmaceutical composition and administered concurrently. In some instances, the IL15R inhibitor and the JAK inhibitor are formulated in two separate pharmaceutical compositions and the two pharmaceutical compositions are administered concurrently. In some embodiments, the IL15R inhibitor and the JAK inhibitor are co-administered substantially simultaneously. In some embodiments, the IL15R inhibitor and the JAK inhibitor are administered sequentially. In some embodiments, the IL15R inhibitor is administered first and the JAK inhibitor is administered second. In some embodiments, the JAK inhibitor is administered first and the IL15R inhibitor is administered second. In some embodiments, the IL15R inhibitor and the JAK inhibitor are administered separately. In some embodiments, the IL15R inhibitor is administered by intravenous injection and the JAK inhibitor is administered orally. In some embodiments, the IL15R inhibitor is administered by intraperitoneal injection and the JAK inhibitor is administered orally. In some embodiments, the IL15R inhibitor is formulated together with a pharmaceutically acceptable excipient, carrier, or dilutant according to a chosen route of administration. In some embodiments, the JAK inhibitor is formulated together with a pharmaceutically acceptable excipient, carrier, or dilutant according to a chosen route of administration.

In some aspects, an IL2R inhibitor and a JAK inhibitor are administered as a combination therapy. In some embodiments, the IL2R inhibitor and the JAK inhibitor are co-administered. In some instances, the IL2R inhibitor and the JAK inhibitor are formulated in a single pharmaceutical composition and administered concurrently. In some instances, the IL2R inhibitor and the JAK inhibitor are formulated in two separate pharmaceutical compositions and the two pharmaceutical compositions are administered concurrently. In some embodiments, the IL2R inhibitor and the JAK inhibitor are co-administered substantially simultaneously. In some embodiments, the IL2R inhibitor and the JAK inhibitor are administered sequentially. In some embodiments, the IL2R inhibitor is administered first and the JAK inhibitor is administered second. In some embodiments, the JAK inhibitor is administered first and the IL2R inhibitor is administered second. In some embodiments, the IL2R inhibitor and the JAK inhibitor are administered separately. In some embodiments, the IL2R inhibitor is administered by intravenous injection and the JAK inhibitor is administered orally. In some embodiments, the IL2R inhibitor is administered by intraperitoneal injection and the JAK inhibitor is administered orally. In some embodiments, the IL2R inhibitor is formulated together with a pharmaceutically acceptable excipient, carrier, or dilutant according to a chosen route of administration. In some embodiments, the JAK inhibitor is formulated together with a pharmaceutically acceptable excipient, carrier, or dilutant according to a chosen route of administration.

In some instances, the IL15R inhibitor and the JAK inhibitor are administered to the subject via the same route of administration (e.g., both systemically, both locally, both intradermally, both cutaneously, both via i.v. injection, etc.). In some instances, the IL15R inhibitor and the JAK inhibitor are administered to the subject via two separate routes of administration (e.g., IL15R inhibitor via i.v. injection and the JAK inhibitor cutaneously or subcutaneously, IL15R inhibitor orally and the JAK inhibitor intradermally, etc.).

In some instances, the IL2R inhibitor and the JAK inhibitor are administered to the subject via the same route of administration (e.g., both systemically, both locally, both intradermally, both cutaneously, both via i.v. injection, etc.). In some instances, the IL2R inhibitor and the JAK inhibitor are administered to the subject via two separate routes of administration (e.g., IL2R inhibitor via i.v. injection and the JAK inhibitor cutaneously or subcutaneously, IL2R inhibitor orally and the JAK inhibitor intradermally, etc.).

In some embodiments, an immunosuppressive agent is administered to the subject. In some embodiments, two or more immunosuppressive agents are administered to the subject. In some embodiments, an immunosuppressive agent is administered to the subject for prophylaxis of aGvHD. In some embodiments, an immunosuppressive agent is administered to the subject for prophylaxis of cGvHD. In some embodiments, an immunosuppressive agent is administered to the subject for treating one or more symptoms of aGvHD. In some embodiments, an immunosuppressive agent is administered to the subject for treating one or more symptoms of cGvHD. In some embodiments, the immunosuppressive agent is administered separately from the IL15R inhibitor. In some embodiments, the immunosuppressive agent is administered separately from the IL2R inhibitor. In some embodiments, the immunosuppressive agent and the IL15R inhibitor are administered sequentially. In some embodiments, the immunosuppressive agent and the IL2R inhibitor are administered sequentially. In some embodiments, the immunosuppressive agent is cyclosporine. In some embodiments, the immunosuppressive agent is tacrolimus. In some embodiments, the immunosuppressive agent is antithymocyte globulin. In some embodiments, the immunosuppressive agent is alemtuzumab. In some embodiments, the immunosuppressive agent is belumosudil. In some embodiments, belumosudil is administered for prophylaxis of cGvHD or for treating cGvHD. In some embodiments, the immunosuppressive agent is ibrutinib. In some embodiments, ibrutinib is administered for prophylaxis of cGvHD or for treating cGvHD. In some embodiments, the immunosuppressive agent is cyclophosphamide. In some embodiments, the immunosuppressive agent is methotrexate. In some embodiments, the immunosuppressive agent is mycophenolate mofetil. In some embodiments, the immunosuppressive agent is sirolimus.

### Treatment Effects

In some instances, the methods of prevention or treatment of cGvHD achieve an effect in the subject. In some embodiments, an effective amount of an IL15R inhibitor is administered to the subject. In some embodiments, an effective amount of an IL15R inhibitor is administered to the subject in need of prevention or treatment of cGvHD. In some embodiments, administering an effective amount of the IL15R inhibitor delays an onset of one or more symptoms of cGvHD in the subject. In some embodiments, administering an effective amount of the IL15R inhibitor alleviates or eliminates one or more symptoms (e.g., frequencies of the symptoms, intensity of the symptoms, etc.) of cGvHD in the subject. In some embodiments, administering an effective amount of the IL15R inhibitor changes one or more molecular markers of cGvHD (e.g., expression, distribution, etc.). In some embodiments, an effective amount of an IL2R inhibitor is administered to the subject. In some embodiments, an effective amount of an IL2R inhibitor is administered to the subject in need of prevention or treatment of cGvHD. In some embodiments, administering an effective amount of the IL2R inhibitor delays an onset of one or more symptoms of cGvHD in the subject. In some embodiments, administering an effective amount of the IL2R inhibitor alleviates or eliminates one or more symptoms (e.g., frequencies of the symptoms, intensity of the symptoms, etc.) of cGvHD in the subject. In some embodiments. administering an effective amount of the IL2R inhibitor changes one or more molecular markers of cGvHD (e.g., expression, distribution, etc.).

In some embodiments, an effective amount of an IL15R inhibitor is administered to the subject in need of prevention or treatment of cGvHD in combination with administering an effective amount of a JAK inhibitor. In some embodiments, the IL15R inhibitor and the JAK inhibitor are administered sequentially, separately, or are co-administered. In some embodiments, administering an effective amount of the IL15R inhibitor in combination with administering an effective amount of the JAK inhibitor delays an onset of one or more symptoms of cGvHD in the subject. In some embodiments, administering an effective amount of the IL15R inhibitor in combination with administering an effective amount of the JAK inhibitor alleviates one or more symptoms of cGvHD in the subject.

In some embodiments, an effective amount of an IL2R inhibitor is administered to the subject in need of prevention or treatment of cGvHD in combination with administering an effective amount of a JAK inhibitor. In some embodiments, the IL2R inhibitor and the JAK inhibitor are administered sequentially, separately, or are co-administered. In some embodiments, administering an effective amount of the IL2R inhibitor in combination with administering an effective amount of the JAK inhibitor delays an onset of one or more symptoms of cGvHD in the subject. In some embodiments, administering an effective amount of the IL2R inhibitor in combination with administering an effective amount of the JAK inhibitor alleviates one or more symptoms of cGvHD in the subject.

In some embodiments, the symptoms of cGvHD to be treated comprise skin rash, raised skin, discolored skin, itchy skin, thickened skin, tightened skin, damaged sweat glands, intolerance to temperature changes, abdominal swelling, yellow discoloration of the eyes, jaundice, elevated or abnormal liver enzyme levels in the blood, dry eyes, changes in vision, dry mouth, white patches in the oral cavity, painful mouth ulcers, pain or sensitivity to hot, cold, spicy, and/or acidic foods, pain or sensitivity to carbonated beverages, shortness of breath, dry cough, chronic cough, wheezing, difficulty breathing, pulmonary changes observed on a chest X-ray, difficulty swallowing, difficulty eating, pain with swallowing, gum disease, tooth decay, loss of appetite, weight loss, nausea, vomiting, diarrhea, stomach pain, fatigue, muscle weakness, muscle cramps, neuromuscular pain, decreased range of motion in joints, decreased range of extension of fingers, wrists, elbows, knees, and/or ankles, tightness in joints or in connective tissue, change in physical activity level, change in locomotor activity level, change in posture, change in gait, vaginal dryness, vaginal itching, vaginal pain, vaginal ulcerations and scarring, narrowing of the vagina, painful vaginal intercourse, narrowing and/or scaring of the urethra, itching and/or scarring of the penis and scrotum, irritation of the penis, change in skin texture, change in skin integrity, scaling of skin, areas of denuded skin, loss of hair on the head, hard nails, brittle nails, nail loss, changes in nail texture, premature graving of the hair, or changes in hair texture, or any combination thereof. In some embodiments, the painful mouth ulcers extend down the throat of the subject. In some embodiments, administering an effective amount of the IL15R inhibitor increases the survival rate of the subject. In some embodiments, administering an effective amount of the IL15R inhibitor in combination with administering an effective amount of the JAK inhibitor increases the survival rate of the subject. In some embodiments, administering an effective amount of the IL15R inhibitor decreases the risk of cGvHD-symptom relapse. In some embodiments, administering an effective amount of the IL15R inhibitor in combination with administering an effective amount of the JAK inhibitor decreases the risk of cGvHD-symptom relapse. In some embodiments, administering an effective amount of the IL2R inhibitor decreases the risk of cGvHD-symptom relapse. In some embodiments, administering an effective amount of the IL2R inhibitor in combination with administering an effective amount of the JAK inhibitor decreases the risk of cGvHD-symptom relapse.

In some embodiments, the methods of prevention or treatment of aGvHD achieve an effect in the subject. In some embodiments, an effective amount of an IL15R inhibitor is administered to the subject in combination with administering an effective amount of a JAK inhibitor. In some embodiments, an effective amount of an IL15R inhibitor is administered to the subject in need of prevention or treatment of aGvHD in combination with administering an effective amount of the JAK inhibitor. In some embodiments, the IL15R inhibitor and the JAK inhibitor are administered sequentially, separately, or are co-administered. In some embodiments, administering an effective amount of the IL15R inhibitor in combination with administering an effective amount of the JAK inhibitor delays an onset of one or more symptoms of aGvHD in the subject. In some embodiments, administering an effective amount of the IL15R inhibitor in combination with administering an effective amount of the JAK inhibitor alleviates one or more symptoms of aGvHD in the subject. In subjects affected by aGvHD, the one or more symptoms of aGvHD may develop within the first 100 days following alloHSCT. In subjects affected by aGvHD, the one or more symptoms of aGvHD may develop after the first 100 days following alloHSCT. In some embodiments, administering an effective amount of the IL15R inhibitor in combination with JAK inhibitor changes one or more molecular markers of aGvHD (e.g., expression, distribution, etc.).

In some embodiments, the methods of prevention or treatment of aGvHD achieve an effect in the subject. In some embodiments, an effective amount of an IL2R inhibitor is administered to the subject in combination with administering an effective amount of a JAK inhibitor. In some embodiments, an effective amount of an IL2R inhibitor is administered to the subject in need of prevention or treatment of aGvHD in combination with administering an effective amount of the JAK inhibitor. In some embodiments, the IL2R inhibitor and the JAK inhibitor are administered sequentially, separately, or are co-administered. In some embodiments, administering an effective amount of the IL2R inhibitor in combination with administering an effective amount of the JAK inhibitor delays an onset of one or more symptoms of aGvHD in the subject. In some embodiments, administering an effective amount of the IL2R inhibitor in combination with administering an effective amount of the JAK inhibitor alleviates one or more symptoms of aGvHD in the subject. In subjects affected by aGvHD, the one or more symptoms of aGvHD may develop within the first 100 days following alloHSCT. In subjects affected by aGvHD, the one or more symptoms of aGvHD may develop after the first 100 days following alloHSCT. In some embodiments, administering an effective amount of the IL2R inhibitor in combination with JAK inhibitor changes one or more molecular markers of aGvHD (e.g., expression, distribution, etc.).

In some embodiments, the symptoms of aGvHD to be treated comprise itchy skin, skin rash, reddened patches on the skin, yellow discoloration of the skin, blisters on the skin, exposed surfaces of the skin flaking off, yellow discoloration of the eyes, jaundice, elevated liver enzyme levels in the blood, nausea, vomiting, diarrhea, abdominal cramping, loss of appetite, or weight loss, or any combination thereof. In some embodiments, the rash on the subject may erupt on the skin of the palms or on the soles of the feet. In some embodiments, the rash on the subject may involve the skin of the trunk or the skin the subject's extremities. In some embodiments, administering an effective amount of the IL15R inhibitor in combination with administering an effective amount of the JAK inhibitor increases the survival rate of the subject. In some embodiments, administering an effective amount of the IL15R inhibitor in combination with administering an effective amount of the JAK inhibitor decreases the risk of aGvHD-symptom relapse. In some embodiments, administering an effective amount of the IL15R inhibitor in combination with administering an effective amount of the JAK inhibitor decreases the risk that the subject develops cGvHD. In some embodiments, administering an effective amount of the IL2R inhibitor in combination with administering an effective amount of the JAK inhibitor increases the survival rate of the subject. In some embodiments, administering an effective amount of the IL2R inhibitor in combination with administering an effective amount of the JAK inhibitor decreases the risk of aGvHD-symptom relapse. In some embodiments, administering an effective amount of the IL2R inhibitor in combination with administering an effective amount of the JAK inhibitor decreases the risk that the subject develops cGvHD.

aGvHD and cGvHD can be graded according to degree and severity of GvHD symptoms for purposes of classification, prognostication, and for assessments of improvement. The Glucksberg-Seattle criteria (GSC) have been in use for over 40 years to classify aGvHD. An updated modified GSC has been recommended for standardized grading of aGvHD. The modified GSC grades aGvHD on a scale of 0) to 4 based on the severity of skin, GI involvement, and liver symptoms. Under the modified GSC, grade () does not indicate aGvHD, grades 1 and 2 are regarded as low-grade aGvHD associated with better outcomes, and grades 3 and 4 are regarded as high-grade aGvHD associated with generally poorer outcomes. Table 1 lists the grading system of the modified Glucksberg-Seattle criteria for assessing aGvHD severity. Additional assessment tools such as the Karnofsky Performance Status Scale can help further inform an extent of functional impairment of the patient and compare effectiveness of different therapies and prognosis of individual patients.

TABLE 1
Modified Glucksberg-Seattle criteria for aGvHD

| | | GI Tract (Diarrhea | | Liver (serum total |
|---|---|---|---|---|
| Grade | Skin (maculopapular rash) | volume) | | bilirubin levels) |
| 0 | No skin involvement | <500 | mL/day | <2 mg/dL |
| 1 | maculopapular rash over less than | 500-1000 | mL/day | 2 to under 3 mg/dL |
| | 25% of body surface area (BSA) | | | |
| 2 | maculopapular rash over 25-50% of | 1000-1500 | mL/day | 3 to under 6 mg/dL |
| | BSA | | | |
| 3 | generalized erythroderma | >1500 | mL/day | 6 to under 13.2 |

| | | | |
|---|---|---|---|
| | | | mg/dL |
| 4 | generalized erythroderma with | Severe abdominal | 13.2 mg/dL and over |
| | bullous formation, often with | pain with or without | |
| | desquamation | ileus or bloody | |
| | | stools | |

The IBMTR grading system of aGvHD groups the patterns of organ involvement into five Indexes (0-D) to be predictive of transplant-related mortality and transplant failure. Index 0 represents no signs of aGvHD in the observed pattern of organ involvement. Criteria for inclusion of IBMTR Severity Index for aGvHD are listed in Table 2. Increasing IBMTR Severity Index is associated with elevated risks of relapse, treatment-related mortality, and treatment failure. The Center for International Blood and Marrow Transplant Research (CIBMTR) has proposed the IBMTR grading system for use in assigning initial aGvHD grades to patients and also assessing progression of aGvHD and response to treatment using IBMTR grading system criteria. The aGvHD grading system proposed for use by the CIBMTR is published in the following publication which is hereby incorporated by reference for descriptions and explanations of aGvHD assessment, grading, classification, and prognosis: (Rowlings P A et al., IBMTR Severity Index for grading acute graft-versus-host disease: retrospective comparison with Glucksberg grade. Br J Haematol. 1997 June:9 (4) 855-64).

TABLE 2
Criteria for IBMTR Severity Index for aGvHD

| | Skin Involvement | | Liver Involvement | | GI Involvement | |
|---|---|---|---|---|---|---|
| | | Extent of | | Total serum | | Volume of |
| | Stage | maculopapular | Stage | bilirubin | Stage | diarrhea |
| Index | (max.) | rash | (max.) | (μmol/L) | (max.) | (mL/day) |
| A | 1 | <25% BSA | 0 | <34 | 0 | <500 |
| B | 2 | 25-50% BSA | 1-2 | 34-102 | 1-2 | 500-1500 |
| C | 3 | >50% BSA | 3 | 103-255 | 3 | >1500 |
| D | 4 | generalized | 4 | >255 | 4 | Severe pain and |
| | | erythroderma | | | | ileus |
| | | with bullous | | | | |
| | | formation | | | | |

The 2014 NIH recommended cGvHD-specific core measurement for assessing response in cGvHD trials system can be used to assess an extent of cGvHD, assess a severity of cGvHD, and determine clinical response to a treatment for cGvHD. This cGvHD grading system is described within the following publication which is hereby incorporated by reference for descriptions and explanations of cGvHD assessment, grading, classification, and prognosis: (Martin P J et al. National Institutes of Health Consensus Development Project on Criteria for Clinical Trials in Chronic Graft-versus-Host Disease: VI. The 2014 Clinical Trial Design Working Group Report. Biol Blood Marrow Transplant. 2015 August:21(8):1343-59.)

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject.” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms of an underlying disorder being treated. Also, a therapeutic benefit can be achieved with the eradication or amelioration of one or more of the physiological symptoms associated with the underlying disorder such that an improvement is observed in the subject. notwithstanding that the subject may still be afflicted with the underlying disorder. A therapeutic benefit may refer to a relieving of one or more symptoms of an underlying disorder being treated or a relieving of the severity of an underlying disorder being treated. The relieving may be a transient effect, a temporary effect, a sustained effect, or a prolonged effect. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “antibody” herein is used in the broadest sense and includes monoclonal antibodies, including intact antibodies and functional (antigen-binding) antibody fragments thereof (also termed antigen-binding fragments thereof), including fragment antigen-binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, tandem di-scFv, tandem tri-scFv. Unless otherwise stated, the term “antibody” should be understood to encompass functional antigen-binding fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region. An antibody includes, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)2, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. A monoclonal antibody can comprise a human IgG1 constant region or a human IgG4 constant region.

The terms “complementarity determining region,” and “CDR,” which are synonymous with “hypervariable region” or “HVR,” are known in the art and refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1,CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991). “Sequences of Proteins of Immunological Interest.” 5th Ed. Public Health Service, National Institutes of Health, Bethesda, MD (“Kabat” numbering scheme). Al-Lazikani et al., (1997) JMB 273.927-948 (“Chothia” numbering scheme); MacCallum et al., J. Mol. Biol. 262:732-745 (1996). “Antibody-antigen interactions: Contact analysis and binding site topography.” J. Mol. Biol. 262. 732-745.” (“Contact” numbering scheme); Lefranc M P et al., “IMGT unique numbering for immunoglobulin and T cell receptor variable domains and Ig superfamily V-like domains.” Dev Comp Immunol. 2003 January;27(1):55-77 (“IMGT” numbering scheme): Honegger A and Plückthun A. “Yet another numbering scheme for immunoglobulin variable domains: an automatic modeling and analysis tool.” J Mol Biol. 2001 Jun. 8;309(3): 657-70. (“Aho” numbering scheme): and Whitelegg N R and Rees A R, “WAM: an improved algorithm for modelling antibodies on the WEB.” Protein Eng. 2000 December;13(12):819-24 (“AbM” numbering scheme. In certain aspects, the CDRs of the antibodies described herein can be defined by a method selected from Kabat, Chothia, IMGT, Aho, AbM, Contact, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example. “30a.” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three CDRs (See e.g., Kindt et al. Kuby Immunology. 6th ed., W. H. Freeman and Co., page 91(2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (See e.g., Portolano et al., J. Immunol. 150:880-887 (1993); Clarkson et al., Nature 352:624-628 (1991)).

Among the described antibodies are antigen-binding fragments thereof. An “antigen-binding fragment thereof” derived from an antibody can refer to a molecule other than an intact antibody that comprises a portion of an intact antibody that binds the antigen to which the intact antibody binds. Examples of antigen-binding fragments thereof derived from an antibody include, but are not limited to, Fv, Fab, Fab′, Fab-SH, F(ab′)2; diabodies; linear antibodies: and single-chain antibody molecules (e.g., scFv or sFv). In particular aspects, the antibodies are single-chain antibody fragments comprising a variable heavy chain region and/or a variable light chain region, such as scFvs. Antigen-binding fragments thereof derived from an antibody can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. The strength, or affinity of immunological binding interactions of antibodies and/or antigen-binding fragments thereof can be expressed in terms of the dissociation constant (K_{d}) of a specific interaction, wherein a smaller K_{d} represents a greater affinity for the antibody or antigen-binding fragment thereof to an antigen. Immunological binding properties of selected polypeptides described herein can be quantified using methods well known in the art. One such method involves measuring the rates of antigen-binding site/antigen complex formation and dissociation, wherein those rates depend on the concentrations of the complex partners, the affinity of the interaction, and various geometric parameters that equally influence the rate in both directions. Thus, both the “on rate constant” (Kₒₙ) and the “off rate constant” (K_{off}) may be determined by calculation of the concentrations and the actual rates of association and dissociation.

A “binding moiety” refers to a portion of a molecule, peptide, polypeptide, antibody, or antigen-binding fragments thereof that mediates specific binding to a recited target or antigen or epitope. By way of example, the binding moiety of an antibody may comprise a heavy-chain/light-chain variable region pair or one or more complementarity determining regions (CDRs).

A “target” as referred to herein refers to the portion of a molecule that participates with a binding moiety of a molecule, peptide, polypeptide, antibody, antibody fragment, or antigen-binding fragment thereof. A target can comprise an amino acid sequence and/or a carbohydrate, lipid, or other chemical entity. An “antigen” is a target comprising a portion that is able to be bound by an adaptive immune molecule such as an antibody or antigen-binding fragment thereof, B-cell receptor, or T-cell receptor.

An “epitope” as described herein refers to the one or more contact regions of an antibody. The contact region of an antibody consists of a discreet number of amino acids contacted by amino acid residues of the antibody (generally CDR residues) and adjacent residues contiguous with the contact residues. For example, the contact region may consist of a continuous stretch of a target protein that is between 5 to 20 amino acids, 5 to 15 amino acids, or 5 to 10 amino acids. This continuous stretch of a target protein may form a linear epitope. An antibody may bind more than one contact region that are separated by 10, 20, 30, 40, 50, 75, or 100 amino acids or more as a result of protein folding. The more than one contact region may form a conformational epitope. Epitopes may be determined using X-ray crystallography, hydrogen-deuterium exchange mass-spec, alanine spanning mutagenesis, competition with excess synthetic peptides as determined by immunoblot, ELISA, surface plasmon radiance, flow cytometry or any other suitable protein binding assay. The epitope may be a functional epitope. The epitope may be a structural epitope.

A “humanized” antibody is an antibody in which all or substantially all CDR amino acid residues are derived from non-human CDRs and all or substantially all FR amino acid residues are derived from human FRs. A humanized antibody optionally can include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

The terms “polypeptide” and “protein” are used interchangeably and refers to a polymer of amino acid residues, and are not limited to a minimum length. Polypeptides, including the provided antibodies and antibody chains and other peptides, e.g., linkers and binding peptides, can include amino acid residues including natural and/or non-natural amino acid residues. The terms also include post-expression modifications of the polypeptide, for example, glycosylation, sialylation, acetylation, phosphorylation, and the like. In some embodiments, the polypeptides can contain modifications with respect to a native or natural sequence, as long as the protein maintains the desired activity. These modifications can be deliberate, as through site-directed mutagenesis, or can be accidental, such as through mutations of hosts which produce the proteins or errors due to PCR amplification.

The section headings used herein are for organizational purposes only and are not to be construed as limiting the subject matter described.

### EXAMPLES

The following examples are included for illustrative purposes only and are not intended to limit the scope of the invention.

### Example 1: Inhibition of IL2-Mediated Proliferation In Vitro by Humanized Anti-CD122 Antibodies

The ability of humanized anti-CD122 antibodies to inhibit proliferation in vitro is tested. A human cell line, TF-1 (ATCC, Manassas, VA), originally established from the bone marrow cells of a subject with erythroleukemia is dependent on the addition of exogenous cytokines such as Erythropoietin (EPO), Granulocyte-macrophage colony-stimulating factor (GM-CSF), or Interleukin-3 (IL3) to culture media for growth. TF-I expresses the common γ chain IL receptor (CD132), but not the IL2/IL15 receptor β chain (CD122). Expression of a CD122-expressing gene in TF-1 allows for the expression of the intermediate and high affinity IL2 and IL15 receptors. TF-1-CD122 cells are generated by transfecting TF-1 with a mammalian expression vector carrying a gene coding for human CD122 and a puromycin resistance gene.

The ability of anti-CD122 monoclonal antibody variants to inhibit cell proliferation mediated by IL2 is examined using TF-CD122 cells. Prior to experiments, TF-CD122 cells are cultured in RPMI 1640 (ThermoFisher Scientific, Cat #11875093) supplemented with 10% heat-inactivated FBS, 50 IU/mL IL2, 2 mM L-glutamine, 50 U/mL penicillin, and 50 μg/mL streptomycin. Cells are maintained at 37° C, under a humidified 5% CO₂ atmosphere. In a typical experiment. TF-1-CD122 cells are first deprived of IL2 for 2 days. After that, about 10⁴ cells per well are incubated with serial dilutions of anti-CD122 monoclonal antibody variants at concentrations between 0.1-10.0 μg/mL. Control wells include three control conditions: 1) no antibody plus 50 IU/mL IL2; 2) InVivoMAb human IgG1 isotype control (BioXCell. NH; Catalog #BE0297) at concentrations matching the serial dilutions: or 3) Human IgG4 kappa (S228P) Isotype Control-CrownVivo™ Antibody (MBL International Corp, MA; Catalog #C0045. Accession #P00698) at concentrations matching the serial dilutions. Treatments with anti-CD122 monoclonal antibody variants (or control conditions) are maintained for 10 minutes at 37° C, and then 50 IU/mL IL2 is added to the culture media. Cells are then cultured for 48 hours. Next, 20 μL per well of alamarBlue™ Cell Viability Reagent (ThermoFisher Scientific; Cat #DAL1025)) is added, and plates are incubated for 6 hours. After the wells are washed. plates are then read using a spectrophotometer microplate reader at 540 and 620 nm. Proliferation curves are obtained following manufacturer's recommendation.

### Example 2: Inhibition of IL15-Mediated Proliferation In Vitro by Humanized Anti-CD122 Antibodies

The ability of humanized anti-CD122 antibodies to inhibit proliferation in vitro is tested. TF-CD122 cells are cultured in RPMI 1640 (ThermoFisher Scientific, Cat #11875093) supplemented with 10% heat-inactivated FBS, 2 mM L-glutamine, 50 U/ml penicillin, and 50 μg/ml streptomycin. To simulate presentation of IL15 in-trans, a soluble complex of human IL15 bound to a portion of the extracellular region of the human IL15Rα (scIL15/IL15Rα) is constructed following the description in Mortier et al., 2006; J. Biol. Chem., 281:1612-1619, and added to cell culture medium at a concentration of 10 nM. Cellular proliferation is assayed to determine if scIL15/IL15Rα at 10 nM can support growth of TF-CD122 cells. Cells are maintained at 37° C, under a humidified 5% CO₂ atmosphere. In a typical experiment. TF-1-CD122 cells are first deprived of scIL15/IL15Rα for 2 days. After that, about 10⁴ cells per well are incubated with serial dilutions of anti-CD122 monoclonal antibody variants at concentrations between 0.1-10.0 μg/mL. Control wells include three control conditions: 1) no antibody plus 10 nM scIL15/IL15Rα; 2) InVivoMAb human IgG1 isotype control (BioXCell, NH; Catalog #BE0297) at concentrations matching the serial dilutions; or 3) Human IgG4 kappa (S228P) Isotype Control-CrownVivo™ Antibody (MBL International Corp, MA; Catalog #C0045, Accession #P00698) at concentrations matching the serial dilutions. Treatments with anti-CD122 monoclonal antibody variants (or control conditions) are maintained for 10 minutes at 37° C, and then 10 nM scIL15/IL15Rα is added to the culture media. Cells are then cultured for 48 hours. Next, 20 μL per well of alamarBlue™ Cell Viability Reagent (ThermoFisher Scientific; Cat #DAL1025)) is added, and plates are incubated for 6 hours. After the wells are washed, plates are then read using a spectrophotometer microplate reader at 540 and 620 nm. Proliferation curves are obtained following manufacturer's recommendation.

### Example 3: Inhibition of IL2 and IL15 Signaling by Anti-CD122 Antibodies

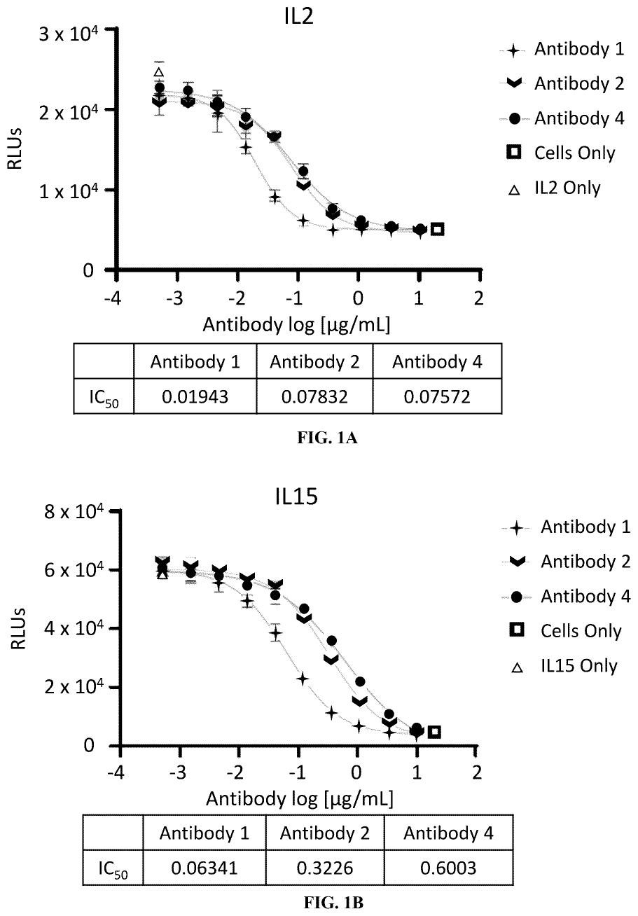

Several anti-CD122 antibodies described herein were tested using in vitro assays for their ability to inhibit IL2 signaling, IL15 signaling, or IL2 and IL15 signaling. A reporter cell line expressing the beta gamma receptor (CD122/CD132) for IL2 and IL15, which has intermediate affinity for each IL2 and IL15, and luciferase under the regulation of IL2 and IL15 binding to its receptor was used to test the ability of anti-CD122 antibodies to IL2 signaling, IL15 signaling, or IL2 and IL15 signaling. In FIG. 1A-FIG. 1B, three anti-CD122 antibodies were assayed for their ability to inhibit IL2 signaling, or IL15 signaling, or IL2 and IL15 signaling. The reporter cells were treated with either IL2 (12.5 ng/mL) or IL15 (6 ng/mL) and also treated with an anti-CD122 antibody at ten different antibody concentrations ranging from 10 μg/mL to 0.5 ng/mL. The anti-CD122 antibody designated as Antibody 4, which is used as a positive control of the experiment, is the anti-human CD122 antibody Clone TU27, obtained from BioLegend®, Catalog #339015. Antibody 1 and Antibody 2 are humanized anti-human CD122 antibodies, each antibody having a different set of CDR sequences. Results from a negative control for these experiments are shown in the graphs in FIG. 1A-FIG. 1B, wherein the cells were treated with either IL2 only (FIG. 1A) or IL15 only (FIG. 1B) without any anti-CD122 antibody co-treatment and the luminescence signals of the cell-based reporter assays were plotted in relative luminescence unit (RLU) values. Results from another negative control for these experiments are shown in the graphs in FIG. 1A-FIG. 1B, wherein the cells were not treated with IL2 or IL15, nor were they treated with anti-CD122 antibody. Measurements of IL2/IL15 signaling after the co-treatment of IL2 or IL15 (12.5 and 6 ng/ml, respectively)) with anti-CD122 antibody (in a dilution series calculated using a dose-response curve, with the tested dose ranging from 10 μg/mL to 1.5 ng/ml) were plotted in RLUs as a function of anti-CD122 antibody concentration (μg/mL in a log scale). The median effective doses (EC50) of IL2 and/or IL15 antagonist (anti-CD122 antibody) for reducing IL2 and/or IL15 signaling in response to IL2 or IL15 treatment were calculated to determine the effects of tested anti-CD122 antibodies. The results in FIG. 1A and FIG. 1B demonstrate that the Antibody 1 antibody is a more potent inhibitor of IL2 signaling and IL15 signaling than Antibody 2 or Antibody 4. Antibody 1 was able in this assay to inhibit both IL2 signaling and IL15 signaling 3-5 fold more efficiently than Antibody 2 and Antibody 4. These results also indicate inhibition of the beta gamma receptor (CD122/CD132) using the tested anti-CD122 antibodies, with Antibody 1 being the most efficient inhibitor.

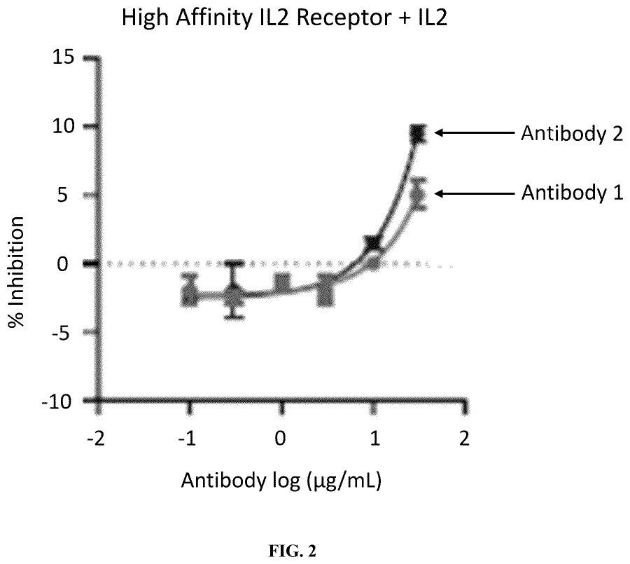

In another in vitro assay, a TF-1αβ cell line expressing the alpha beta gamma receptor (IL2Rα/IL2Rβ/IL2Rγ complex) for IL2, which has high affinity for IL2, was used to test the ability anti-CD122 antibodies to inhibit IL2-mediated cell proliferation. In FIG. 2, cells were treated with IL2 at a dosage of 10 ng/mL and also were treated with an anti-CD122 antibody at six different antibody concentrations ranging from 30 μg/mL to 30 ng/ml. The results were plotted as a percentage of inhibition in cell proliferation compared to no antibody treatment over concentration of anti-CD122 antibody. The plotted data indicates that neither Antibody 2 nor Antibody 1 are efficient inhibitors of the IL2 alpha beta gamma receptor.

### Example 4: Acute GvHD Study for Dose-ranging Investigation of Antibody 1 and Antibody 2 for Prophylaxis

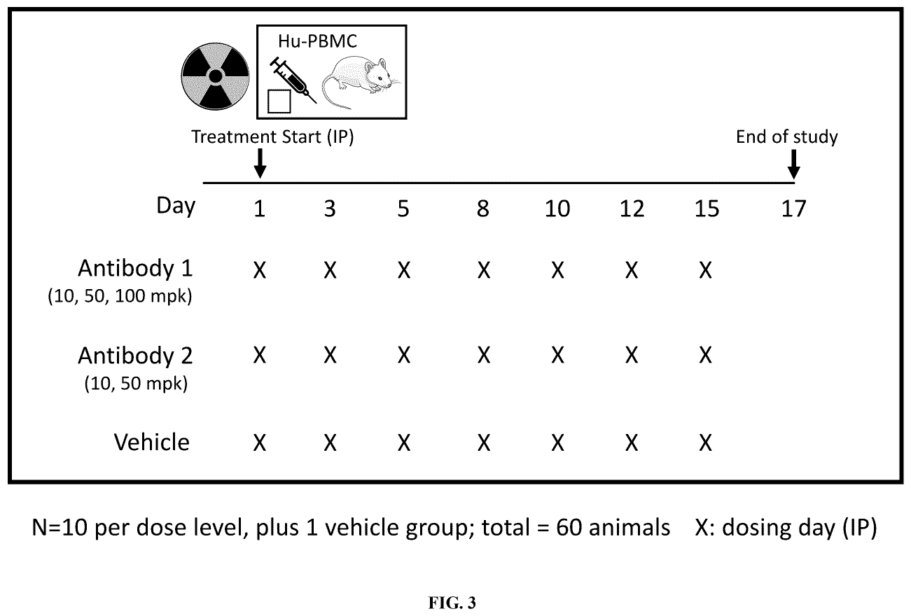

A xenograft GvHD mouse model was utilized to study human T and NK cell-mediated aGvHD and the therapeutic effects of anti-CD122 antibody treatment therein. Female NCG-hIL-15 mice were irradiated at a dosage of 100 cGy on Day 1 of the protocol and human peripheral blood mononuclear cells (Hu-PBMCs) were transplanted via injection at a dosage of 1×10⁷ cells/200 μL/mice via IV administration into the irradiated mice. PBMCs are immune cells that originate in the bone marrow and are secreted into the peripheral circulation forming critical components of the immune system. PBMCs are involved in humoral and cell-mediated immune function. As seen from the study design in FIG. 3, treatment with anti-CD122 antibodies was also begun on Day 1 prior to any potential onset of aGvHD symptoms. Groups of 10 mice were used for each test category. A vehicle group (n=10) served as a negative control with no treatment administered following transplant. Three groups of Antibody 1 treated animals (n=10 for each group) were tested using a) 10 milligram per kilogram of body weight (mpk) of antibody, b) 50 mpk of antibody, and c) 100 mpk of antibody, respectively. Two groups of Antibody 2 treated animals (n=10 for each group) were tested using a) 10 mpk of antibody or b) 50) mpk of antibody. Animals were treated via intraperitoneal (IP) administration of either Antibody 1 or Antibody 2 on D1, D3, D5, D8, D10, D12 and D15. The study concluded on Day 17. Treatment using Antibody 1 or Antibody 2 at various test dosages between the range of 10-150) mpk of antibody are also undertaken to assess treatment effectiveness. Antibody 1 and Antibody 2 are humanized anti-human CD122 antibodies, each antibody having a different set of CDR sequences.

An aggressive form of aGvHD was established with this protocol and the effects on prophylaxis using anti-CD122 antibody treatment are determined. Vehicle-treated mice had 100% mortality by Day 15. Results of different Antibody 1 and Antibody 2 treatment dosages on survival at study conclusion are determined. Results are analyzed by statistical analysis using Fisher's exact test indicating a statistically significant improved result in survival rate of subjects treated with a particular dosage of either Antibody 1 or Antibody 2 compared with vehicle.

From these studies, a therapeutic benefit with a method of treatment using an anti-CD122 antibody as a prophylaxis against aGvHD is evident.

These results demonstrate that anti-CD122 antibody therapy is an effective therapeutic strategy for aGvHD.

### Example 5: Acute GvHD Study Comparing Single and Combination Therapies

A xenograft GvHD mouse model was utilized to study human T and NK cell-mediated aGvHD and the therapeutic effects of anti-CD122 antibody treatment in single or combination therapy approaches. Female NCG-hIL-15 mice were irradiated at a dosage of 100 cGy on Day 1 of the protocol and human peripheral blood mononuclear cells (PBMCs) were transplanted via injection at a dosage of 1×10⁷ cells/200 μL/mice via IV administration into the irradiated mice according to the same xenograft GvHD mouse model design as in Example 4.

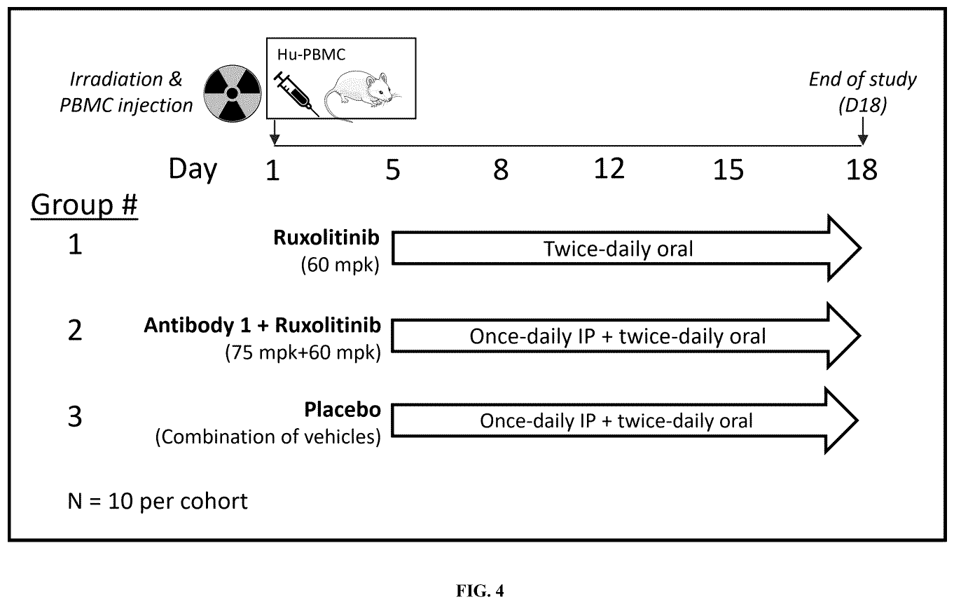

Treatment according to 3 different grouping of animals was begun on Day 5 following transplant, as shown in the study design of FIG. 4. Groups of 10 mice were used for each test category. A single treatment group, Group #1, (n=10) received ruxolitinib (at a dosage of 60 mpk) twice-daily by oral administration (PO) beginning on Day 5 and ending on Day 18. Days of treatment were D5, D6, D7, D8, D9, D10, D11, D12, D13, D14, D15, D16, D17, and D18. A combination therapy group. Group #2. (n=10) received ruxolitinib (at a dosage of 60 mpk) twice-daily by oral administration beginning on Day 5 and ending on Day 18. Days of treatment using ruxolitinib in Group #2 were D5, D6, D7, D8, D9, D10, D11, D12, D13, D14, D15, D16,D17, and D18. Group #2 animals separately received Antibody 1 therapy (at a dosage of 75 mpk) once-daily by IP administration. Days of treatment using Antibody 1 in Group #2 were D5, D6, D7, D8, D9, D10, D11, D12, D13, D14, D15, D16, D17, and D18. A placebo group, Group #3, (n=10) serves as a negative control with vehicle-only administered following transplant received twice daily oral placebo (vehicle only) beginning on Day 5 and ending on Day 18. Days of oral placebo treatment were D5, D6, D7, D8, D9, D10, D11, D12, D13, D14,D15, D16, D17, and D18. This vehicle group also separately received once daily IP placebo (vehicle only) on D5, D6, D7, D8, D9, D10, D11, D12, D13, D14, D15, D16, D17, and D18.

Day 18 was the end of study day for all treatment groups. Animals from all treatment groups were assessed daily beginning on Day 1 and ending on Day 18 for survival following transplant. A comparison of survival rate between Group #1 (single treatment group with ruxolitinib monotherapy), Group #2 (combination therapy group with ruxolitinib and Antibody 1 combination treatment), and Group #3 (placebo group serving as negative control) was undertaken to evaluate treatment effects on subject survival rate daily throughout the study and to evaluate overall survival rate at the end of the study. At the end of the study, tissue was collected from all surviving animals for histological assessment of key target organs. These key target organs include liver, lung, and skin. A histological and phenotypic analysis of collected tissue is undertaken to determine the presence and extent of aGvHD symptoms at an organ level, a tissue level, a cellular level, and a molecular level. A comparison of histological findings between treatment groups is undertaken to determine which treatment groups yield the most favorable outcomes.

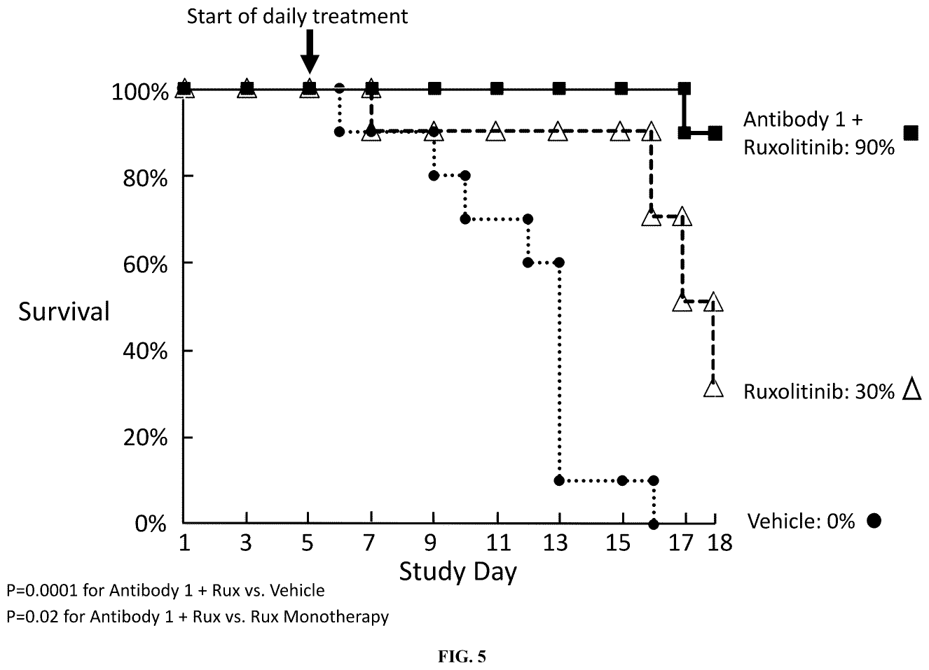

The results indicate a therapeutic benefit for survival in this model of aGvHD for animals receiving combination therapy of ruxolitinib treatment and Antibody 1 treatment compared with either ruxolitinib single therapy or compared with placebo-treatment. FIG. 5 shows results from this comparison study. Survival for treatment group #1, #2, and #3 was assessed daily and plotted to assess survival duration and compare by treatment group. At the end of the study, Group #2 (Antibody 1+ruxolitinib) animals had a 90% survival rate. Group #1 (ruxolitinib) animals had a 30% survival rate, and Group #3 (Vehicle-treated control; termed ·Vehicle') animals had a 0% survival rate. Statistical analysis using Fisher's exact test indicated that combination therapy of ruxolitinib treatment and Antibody 1 treatment yielded a statistically significant improved result in survival rate of subjects compared with ruxolitinib single therapy (p-value=0.02). Statistical analysis using Fisher's exact test indicated that combination therapy of ruxolitinib treatment and Antibody 1 treatment yielded a statistically significant improved result in survival rate of subjects compared with placebo-treatment (p-value=0.0001). Combination therapy for aGvHD comprising administering anti-CD122 antibody and ruxolitinib demonstrated a drastic improvement in survival rate compared with ruxolitinib monotherapy. This result may indicate a synergistic effect of targeting the IL2, IL15, or IL2 and IL15 signaling pathways at different levels of the signal transduction cascade (e.g., targeting IL2 and/or IL15 signal reception and/or initiation of transduction with treatment using anti-CD122 antibody and targeting downstream signal transduction through modulating protein phosphorylation by inhibiting Janus kinases to ultimately yield a more robust effect of modulating IL2 and/or IL15 signal transduction responses in cells, tissues, and organs subjected to the immunological and pathology effects of aGvHD). This result indicates that combination treatment of IL2R inhibitor therapy, IL15R inhibitor therapy, or IL2R/IL15R inhibitor therapy combined with JAK inhibitor therapy can improve survival outcome in an aGvHD model compared with other single treatments tested.

### Example 6: Chronic GvHD Study to Investigate Anti-CD122 Antibody Monotherapy in Comparison With a JAK Inhibitor

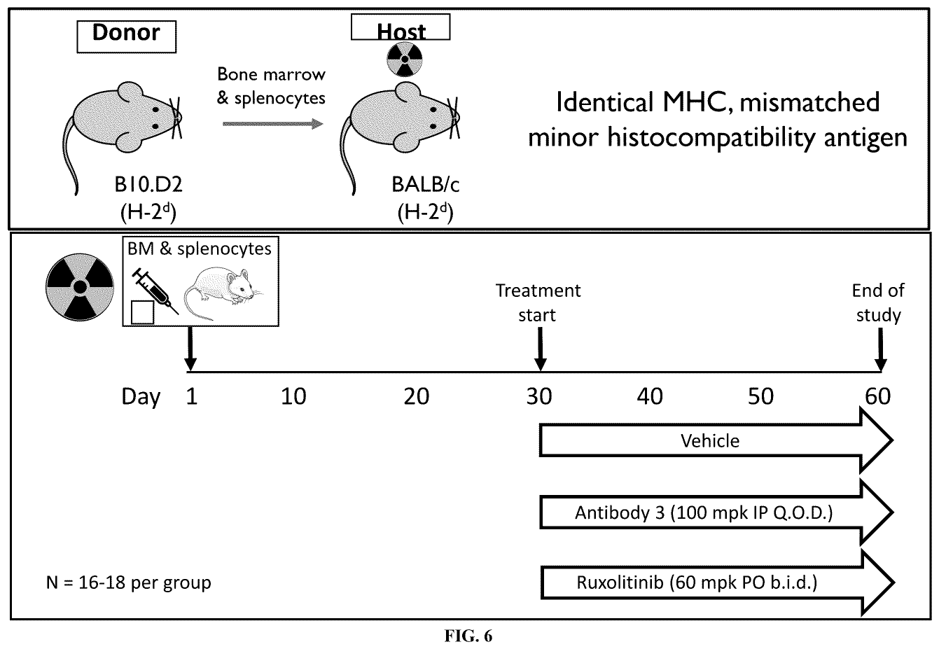

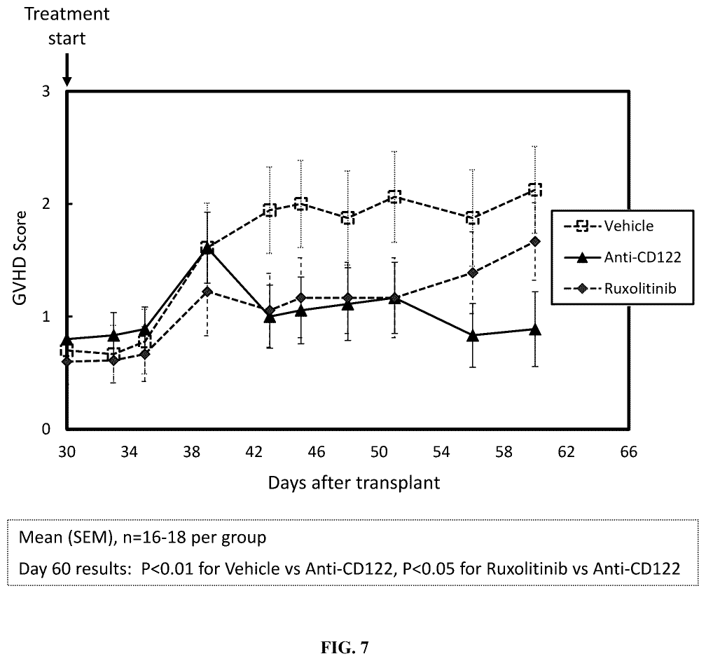

An allogeneic model of chronic GvHD according to the animal model design of Ramos et al (Ramos T L et al. Delayed administration of ixazomib modifies the immune response and prevents chronic graft-versus-host disease. Bone Marrow Transplant. 2021 December;56(12):3049-3058) was investigated. In this model as shown in the study design of FIG. 6. B10.D2 mice serve as donor for bone marrow and splenocytes to be transplanted into irradiated BALB/c host mice. Both of these mouse strains possess identical MHC (H-24), but are mismatched for minor histocompatibility antigens. In this model of cGvHD, there is a low mortality rate extending past 150 days post-transplant and cGvHD symptoms can typically appear starting at approximately 30 days post-transplant. Study design includes treatment start at Day 30 post-transplant and end of study is marked at Day 60 post-transplant. Each group had an N=16-18 animals tested. Two test treatment groups included Group 2) Antibody 3 anti-CD122 antibody therapy (100 mpk delivered IP every other day (Q.O.D.), and Group 3) ruxolitinib monotherapy (60 mpk PO b.i.d). One negative control group (Group 1) including vehicle (IP Q.O.D.)+vehicle (PO b.i.d.) was also tested. Phenotypic cGvHD score and survival were assessed starting at Day 30 and ending at Day 60 post-transplant. FIG. 7 shows a graph of mean GvHD score and error bars representing standard error of the mean assessed at various time points from initiation of treatment (Day 30) to end of treatment (Day 60). Results indicate that anti-CD122 antibody monotherapy treatment via administration of Antibody 3 anti-CD122 antibody produces superior therapeutic effects on cGvHD symptoms than placebo treatment or ruxolitinib monotherapy (Day 60 Antibody 3 vs Vehicle p value<0.01: Day 60 Antibody 3 vs Ruxolitinib p value<0.05).

Following this phenotypic analysis, histological assessment of key target organs (including liver, lung, and skin) is undertaken in all surviving animals to confirm cGvHD pathology and provide a separate measurement of prophylaxis of cGvHD pathology or improvement of cGvHD pathology for each treatment group.

Detailed experiments protocols for testing this cGvHD model are listed below:

### Drugs and Treatment

| Group | N | Strain | Active Agent | dose | Route | Schedule |
|---|---|---|---|---|---|---|
| 1 | 16 | BALB/c | None | None | IP and PO | IP Q.O.D. and PO |
| | | | (Vehicles only) | | | b.i.d. |
| 2 | 18 | BALB/c | Antibody 3 | 100 mpk | IP | every other day |
| | | | | | | (Q.O.D.) |
| 3 | 18 | BALB/c | Ruxolitinib | 60 mpk | PO | 2x/day (b.i.d.) |

### Procedures

BALB/c mice were randomized into treatment groups based on Day 1 bodyweight.
Female BALB/c mice were irradiated (5 Gy) and intravenously injected with B10.D2 donor cells on Day 1.
Cell injection volume was about 200 μL of Bone Marrow cells and splenocytes mixture as described below in treatments. Total cell volume injection=200 μL in PBS.
GvHD Scoring and body weight measurement assessed on Day 30, 33, 35, 37, 39, 41, 43, 45, 48, 51, 53, 56, 58, and 60.
Individual scores according to the grading system below of Grade 0, 1, or 2 were measured and documented on each assessed day for each criterion of body weight loss, activity, posture, fur texture, and skin integrity for each subject in each test group. Each criterion was then summed per subject to yield a total GvHD score by subject per assessed day (e.g., add score for bodyweight loss, activity, posture, fur texture, and skin integrity to yield total GvHD score).
Total GvHD scores per animal per day were then averaged per group to yield average GvHD score per test group and the standard error of the mean (SEM) for each group was determined.

GvHD Scoring:

| Criteria | Grade 0 | Grade 1 | Grade 2 |
|---|---|---|---|
| Weight Loss | <10% | ≥10%, <25% | ≥25% |
| Activity | Normal | Mild to moderately | Stationary until |
| Posture | Normal | Hunching only at rest | Severe gait, impaired |
| Fur Texture | Normal | Mild to moderate | Severe ruffling/poor |
| Skin Integrity | Normal | Obvious areas of | Scaling of paws |
| | | denuded skin | and/or tail |

Average total GvHD scores per assessed day for Group #1, 2, and 3, and the corresponding SEM values are listed in Table 3.

TABLE 3
Average total GvHD scores and SEM values by treatment group

| | Days: | | | | | | | | | |
|---|---|---|---|---|---|---|---|---|---|---|
| | D 30 | D 33 | D 35 | D 39 | D 43 | D 45 | D 48 | D 51 | D 56 | D 60 |
| Group # | mean | | | | | | | | | |
| 1 | 0.7 | 0.7 | 0.8 | 1.6 | 1.9 | 2.0 | 1.9 | 2.1 | 1.9 | 2.1 |
| 2 | 0.8 | 0.8 | 0.9 | 1.6 | 1.0 | 1.1 | 1.1 | 1.2 | 0.8 | 0.9 |
| 3 | 0.6 | 0.6 | 0.7 | 1.2 | 1.1 | 1.2 | 1.2 | 1.2 | 1.4 | 1.7 |
| Group # | SEM | | | | | | | | | |
| 1 | 0.3 | 0.3 | 0.3 | 0.4 | 0.4 | 0.4 | 0.4 | 0.4 | 0.4 | 0.4 |
| 2 | 0.2 | 0.2 | 0.2 | 0.3 | 0.3 | 0.3 | 0.3 | 0.3 | 0.3 | 0.3 |
| 3 | 0.2 | 0.2 | 0.2 | 0.4 | 0.3 | 0.4 | 0.3 | 0.4 | 0.4 | 0.3 |

Histological assessment of key target organs, including liver, lung, and skin, are conducted in all surviving animals at study termination.

While preferred aspects of the present invention have been shown and described herein, it will be obvious to those skilled in the art that such aspects are provided by way of example only. Numerous variations, changes, and substitutions will now occur to those skilled in the art without departing from the invention. It should be understood that various alternatives to the aspects of the invention described herein may be employed in practicing the invention. It is intended that the following claims define the scope of the invention and that methods and structures within the scope of these claims and their equivalents be covered thereby.

### ASPECTS

Aspect 1: A method of preventing or treating chronic graft versus host disease (cGvHD) in a subject in need thereof, the method comprising: administering to the subject an effective amount of an IL2 receptor (IL2R) inhibitor, thereby preventing or treating cGvHD in the subject.

Aspect 2: The method of aspect 1, wherein the IL2R inhibitor is a small molecule inhibitor, an antibody, or its antigen-binding fragment thereof, a peptide inhibitor, or nucleotide-based inhibitor.

Aspect 3: The method of aspect 1 or 2, wherein the IL2R inhibitor is a CD122 inhibitor.

Aspect 4: The method of any one of aspects 1-3, wherein the IL2R inhibitor interferes with an IL2 binding to the IL2R.

Aspect 5: The method of any one of aspects 1-4, wherein the IL2R inhibitor disrupts or diminishes: a) IL2-induced signal transduction, b) CD122-mediated signal transduction, or c) IL2 from binding to an IL2Rβ/IL2Rγ complex, or any combination thereof.

Aspect 6: The method of any one of aspects 1-5, wherein the effective amount of the IL2R inhibitor does not significantly disrupt IL2 from binding to a high affinity IL2Rα/IL2Rβ/IL2Rγ complex or does not significantly disrupt IL2 from signaling through a high affinity IL2Rα/IL2Rβ/IL2Rγ complex.

Aspect 7: The method of any one of aspects 1-6, wherein an IL2-stimulated Janus kinase (JAK) signaling pathway is disrupted or inhibited in cells of the subject receiving the effective amount of the IL2R inhibitor.

Aspect 8: The method of any one of aspects 2-7, wherein the IL2R inhibitor is an antibody.

Aspect 9: The method of any one of aspects 2-8, wherein the antibody is an anti-CD122 antibody.

Aspect 10: The method of aspect 9, wherein the anti-CD122 antibody is a monoclonal antibody.

Aspect 11: The method of any one of aspects 2-10, wherein the antibody is a human antibody, a humanized antibody, or a chimeric antibody.

Aspect 12: The method of any one of aspects 2-11, wherein the antibody comprises an Immunoglobulin G (IgG) antibody or variant thereof.

Aspect 13: The method aspect 12, wherein the IgG antibody or variant thereof comprises an IgG1 antibody or variant thereof, an IgG2 antibody or variant thereof, an IgG3 antibody or variant thereof, or an IgG4 antibody or variant thereof.

Aspect 14: The method of any one of aspects 2-13, wherein the antibody or its antigen-binding fragment thereof comprises IgG-scFv, IgA, IgM, IgE antibody, nanobody, mini-antibody, minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFy-Fc, or intrabody.

Aspect 15: The method of any one of aspects 9-14, wherein the anti-CD122 antibody interferes with IL2 binding to the IL2R.

Aspect 16: The method of any one of aspects 9-15, wherein the anti-CD122 antibody diminishes or disrupts IL2-induced signal transduction.

Aspect 17: The method of any one of aspects 9-16, wherein the anti-CD122 antibody diminishes or disrupts IL2-induced signal transduction through the intermediate affinity IL-βγ receptor.

Aspect 18: The method of any one of aspects 9-17, wherein the anti-CD122 antibody diminishes or disrupts IL2-induced signal transduction through the intermediate affinity IL-βγ receptor, and wherein IL2-induced signal transduction through the high affinity IL-αβγ receptor is not significantly diminished or disrupted.

Aspect 19: The method of any one of aspects 1-18, wherein the administering an effective amount of the IL2R inhibitor delays an onset of one or more symptoms of cGvHD in the subject.

Aspect 20: The method of any one of aspects 1-19, wherein the administering an effective amount of the IL2R inhibitor alleviates one or more symptoms of cGvHD in the subject.

Aspect 21: The method of any one of aspects 1-20, wherein the administering an effective amount of the IL2R inhibitor significantly relieves cGvHD severity in the subject.

Aspect 22: The method of any one of aspects 1-21, wherein the administering improves an objective response rate (ORR) at 6 months compared to a first-line standard of care therapy, a second-line standard of care therapy, or a third-line standard of care therapy.

Aspect 23: The method of aspect 22, wherein the first-line standard of care therapy comprises treatment with one or more corticosteroids.

Aspect 24: The method of aspect 22, wherein the first-line standard of care therapy comprises treatment with one or more JAK inhibitors.

Aspect 25: The method of aspect 22, wherein the first-line standard of care therapy comprises treatment with ruxolitinib.

Aspect 26: The method of any one of aspects 22-25, wherein the second-line standard of care therapy comprises treatment with either ruxolitinib or ibrutinib.

Aspect 27: The method of any one of aspects 22-26, wherein the second-line standard of care therapy comprises treatment with one or more corticosteroids.

Aspect 28: The method of any one of aspects 22-27, wherein the third-line standard of care therapy comprises treatment with belumosudil.

Aspect 29: The method of any one or aspects 19-28, wherein the one or more symptoms of cGvHD comprises skin rash, raised skin, discolored skin, itchy skin, thickened skin, tightened skin, damaged sweat glands, intolerance to temperature changes, abdominal swelling, yellow discoloration of the eyes, jaundice, elevated or abnormal liver enzyme levels in the blood, dry eyes, changes in vision, dry mouth, white patches in the oral cavity, painful mouth ulcers, pain or sensitivity to hot, cold, spicy, and/or acidic foods, pain or sensitivity to carbonated beverages, shortness of breath, dry cough, chronic cough, wheezing, difficulty breathing, pulmonary changes observed on a chest X-ray, difficulty swallowing, difficulty eating, pain with swallowing, gum disease, tooth decay, loss of appetite, weight loss, nausea, vomiting, diarrhea, stomach pain, fatigue, muscle weakness, muscle cramps, neuromuscular pain, decreased range of motion in joints, decreased range of extension of fingers, wrists, elbows, knees, and/or ankles, tightness in joints or in connective tissue, change in physical activity level, change in locomotor activity level, change in posture, change in gait, vaginal dryness, vaginal itching, vaginal pain, vaginal ulcerations and scarring, narrowing of the vagina, painful vaginal intercourse, narrowing and/or scaring of the urethra, itching and/or scarring of the penis and scrotum, irritation of the penis, change in skin texture, change in skin integrity, scaling of skin, areas of denuded skin, loss of hair on the head, hard nails, brittle nails, nail loss, changes in nail texture, premature graving of the hair, or changes in hair texture, or any combination thereof.

Aspect 30: The method of any one of aspects 1-29, wherein the administering an effective amount of the IL2R inhibitor: a) reduces expression of one or more biomarkers of cGvHD in the subject, b) increases the survival rate of the subject, or c) decreases a risk of cGvHD-symptom relapse in the subject, or any combination thereof.

Aspect 31: The method of any one of aspects 1-30, wherein the IL2R inhibitor is administered systemically, locally, intradermally, subcutaneously, or topically.

Aspect 32: The method of aspect 31, wherein the IL2R inhibitor administered systemically is administered by intravenous injection, by enteral administration, or through inhalation.

Aspect 33: The method of aspect 32, wherein the IL2R inhibitor administered by enteral administration is administered orally.

Aspect 34: A method of preventing or treating GvHD in a subject in need thereof, the method comprising: administering to the subject an effective amount of i) an IL2 receptor (IL2R) inhibitor and ii) a JAK inhibitor, thereby preventing or treating GvHD in the subject.

Aspect 35: The method of aspect 34, wherein the GvHD is acute graft versus host disease (aGvHD).

Aspect 36: The method of aspect 35, wherein the aGvHD is steroid refractory aGvHD.

Aspect 37: The method of aspect 35, wherein the aGvHD is JAK inhibitor refractory aGvHD.

Aspect 38: The method of aspect 35, wherein the aGvHD is ruxolitinib refractory aGvHD.

Aspect 39: The method of aspect 34, wherein the GvHD is chronic graft versus host disease (cGvHD).

Aspect 40: The method of any one of aspects 34-39, wherein the IL2R inhibitor and the JAK inhibitor are co-administered.

Aspect 41: The method of any one of aspects 34-40, wherein the IL2R inhibitor and the JAK inhibitor are administered separately or sequentially.

Aspect 42: The method of any one of aspects 34-41, wherein the JAK inhibitor is selected from the group consisting of ruxolitinib, abrocitinib, baricitinib, delgocitinib, fedratinib, filgotinib, oclacitinib, pacritinib, peficitinib, tofacitinib, itacitinib and upadacitinib.

Aspect 43: The method of any one of aspects 34-42, wherein the JAK inhibitor is ruxolitinib.

Aspect 44: The method of any one of aspects 34-43, wherein the IL2R inhibitor is a CD122 inhibitor.

Aspect 45: The method of any one of aspects 34-44, wherein the IL2R inhibitor is an antibody or its antigen-binding fragment thereof, a small molecule inhibitor, a peptide inhibitor, or a nucleotide-based inhibitor.

Aspect 46: The method of aspect 45, wherein the antibody is a human antibody, a humanized antibody, or a chimeric antibody.

Aspect 47: The method of aspect 45 or 46, wherein the antibody comprises an Immunoglobulin G (IgG) antibody or variant thereof.

Aspect 48: The method of aspect 47, wherein the IgG antibody or variant thereof comprises an IgG1 antibody or variant thereof, an IgG2 antibody or variant thereof, an IgG3 antibody or variant thereof, or an IgG4 antibody or variant thereof.

Aspect 49: The method of any one of aspects 45-48, wherein the antibody or its antigen-binding fragment thereof comprises IgG-scFv, IgA, IgM, IgE antibody, nanobody, mini-antibody, minibody, scFv-CH3 KIH, Fab-scFv-Fc KIH, Fab-scFv, scFv-CH-CL-scFv, Fab ′, F(ab′)2, F(ab′)3,_F(ab′)2-scFv2, scFv, scFv-KIH, Fab-scFv-Fc, or intrabody.

Aspect 50: The method of any one of aspects 34-49, wherein the IL2 receptor inhibitor is an anti-CD122 antibody.

Aspect 51: The method of any one of aspects 34-50, wherein the JAK inhibitor is administered first and the IL2R inhibitor is administered second.

Aspect 52: The method of any one of aspects 34-51, wherein the administering an effective amount of the IL2R inhibitor and the JAK inhibitor delays an onset of one or more symptoms of aGvHD or cGvHD in the subject.

Aspect 53: The method of any one of aspects 34-52, wherein the administering an effective amount of the IL2R inhibitor and the JAK inhibitor alleviates one or more symptoms of aGvHD or cGvHD in the subject.

Aspect 54: The method of aspect 52 or 53, wherein the one or more symptoms of aGvHD comprises itchy skin, skin rash, reddened patches on the skin, yellow discoloration of the skin, blisters on the skin, exposed surfaces of the skin flaking off, yellow discoloration of the eyes, jaundice, elevated liver enzyme levels in the blood, nausea, vomiting, diarrhea, abdominal cramping, loss of appetite, or weight loss, or any combination thereof.

Aspect 55: The method of aspect 52 or 53, wherein the one or more symptoms of cGvHD comprises skin rash, raised skin, discolored skin, itchy skin, thickened skin, tightened skin, damaged sweat glands, intolerance to temperature changes, abdominal swelling, yellow discoloration of the eyes, jaundice, elevated or abnormal liver enzyme levels in the blood, dry eyes, changes in vision, dry mouth, white patches in the oral cavity, painful mouth ulcers, pain or sensitivity to hot, cold, spicy, and/or acidic foods, pain or sensitivity to carbonated beverages, shortness of breath, dry cough, chronic cough, wheezing, difficulty breathing, pulmonary changes observed on a chest X-ray, difficulty swallowing, difficulty eating, pain with swallowing, gum disease, tooth decay, loss of appetite, weight loss, nausea, vomiting, diarrhea, stomach pain, fatigue, muscle weakness, muscle cramps, neuromuscular pain, decreased range of motion in joints, decreased range of extension of fingers, wrists, elbows, knees, and/or ankles, tightness in joints or in connective tissue, change in physical activity level, change in locomotor activity level, change in posture, change in gait, vaginal dryness, vaginal itching, vaginal pain, vaginal ulcerations and scarring, narrowing of the vagina, painful vaginal intercourse, narrowing and/or scaring of the urethra, itching and/or scarring of the penis and scrotum, irritation of the penis, change in skin texture, change in skin integrity, scaling of skin, areas of denuded skin, loss of hair on the head, hard nails, brittle nails, nail loss, changes in nail texture, premature graying of the hair, or changes in hair texture, or any combination thereof.

Aspect 56: The method of any one of aspects 34-55, wherein the administering an effective amount of the IL2R inhibitor and the JAK inhibitor reduces expression of one or more biomarkers of aGvHD or cGvHD in the subject.

Aspect 57: The method of any one of aspects 34-56, wherein the administering an effective amount of the IL2R inhibitor and the JAK inhibitor increases the survival rate of the subject.

Aspect 58: The method of any one of aspects 34-57, wherein the administering an effective amount of the IL2R inhibitor and the JAK inhibitor increases the survival rate of the subject compared with a subject treated with a JAK inhibitor as a monotherapy.

Aspect 59: The method of any one of aspects 34-58, wherein the administering an effective amount of the IL2R inhibitor and the JAK inhibitor decreases a risk of aGvHD-symptom relapse or cGvHD-symptom relapse.

Aspect 60: The method of any one of aspects 34-59, wherein the IL2R inhibitor and the JAK inhibitor are administered by the same route of administration.

Aspect 61: The method of any one of aspects 34-60, wherein the IL2R inhibitor and the JAK inhibitor are administered by separate routes of administration.

Aspect 62: The method of any one of aspects 34-61, wherein the IL2R inhibitor is administered systemically, locally, intradermally, subcutaneously, or topically, and wherein the JAK inhibitor is administered systemically, locally, intradermally, subcutaneously, or topically.

Aspect 63: The method of aspect 62, wherein the IL2R inhibitor administered systemically is administered by intravenous injection, by subcutaneous injection, by enteral administration, or through inhalation.

Aspect 64: The method of aspect 62 or 63, wherein the JAK inhibitor administered systemically is administered by intravenous injection, by subcutaneous injection, by enteral administration, or through inhalation.

Aspect 65: The method of aspect 63 or 64, wherein the IL2R inhibitor administered by enteral administration is administered orally.

Aspect 66: The method of aspect 64 or 65, wherein the JAK inhibitor administered by enteral administration is administered orally.

Aspect 67: The method of any one of aspects 34-66, wherein the administering an effective amount of the IL2R inhibitor and the JAK inhibitor significantly prevents development of an extent of cGvHD severity in the subject.

Aspect 68: The method of any one of aspects 34-67, wherein the administering an effective amount of the IL2R inhibitor and the JAK inhibitor significantly relieves GvHD severity.

Aspect 69: The method of aspect 68, wherein the administering improves an ORR at 28 day's compared to treatment comprising ruxolitinib monotherapy.

Aspect 70: The method of any one of aspects 34-69, wherein the administering significantly prevents development of an extent of cGvHD severity in the subject according to a total cGvHD assessment compared to placebo treatment, a first-line standard of care cGvHD therapy, a second-line standard of care cGvHD therapy, or a third-line standard of care cGvHD therapy.

Aspect 71: The method of aspect 70, wherein the first-line standard of care therapy comprises treatment with one or more corticosteroids.

Aspect 72: The method of aspect 70, wherein the first-line standard of care therapy comprises treatment with one or more JAK inhibitors.

Aspect 73: The method of aspect 70, wherein the first-line standard of care therapy comprises treatment with ruxolitinib.

Aspect 74: The method of any one of aspects 70-73, wherein the second-line standard of care therapy comprises treatment with either ruxolitinib or ibrutinib.

Aspect 75: The method of any one of aspects 70-74, wherein the second-line standard of care therapy comprises treatment with one or more corticosteroids

Aspect 76: The method of any one of aspects 70-75, wherein the third-line standard of care therapy comprises treatment with belumosudil.

Aspect 77: The method of any one of aspects 34-76, wherein the administering significantly prevents development of an extent of cGvHD severity in the subject according to a total cGvHD assessment compared to treatment comprising ruxolitinib monotherapy.

Aspect 78: A method of preventing or treating chronic graft versus host disease (cGvHD) in a subject in need thereof, the method comprising: administering to the subject an effective amount of an IL2 receptor (IL2R)/IL15 receptor (IL15R) inhibitor, thereby preventing or treating cGvHD in the subject.

Aspect 79: The method of aspect 78, wherein the IL2R/IL15R inhibitor is a small molecule inhibitor, an antibody, or its antigen-binding fragment thereof, a peptide inhibitor, or nucleotide-based inhibitor.

Aspect 80: The method of aspect 78 or 79, wherein the IL2R/IL15R inhibitor is a CD122 inhibitor.

Aspect 81: The method of any one of aspects 78-80, wherein the IL2R/IL15R inhibitor interferes with an IL2 binding to the IL2R and with an IL15 binding to the IL15R.

Aspect 82: The method of any one of aspects 78-81, wherein the IL2R/IL15R inhibitor disrupts or diminishes: a) IL2-induced signal transduction and IL15-induced signal transduction, b) CD122-mediated signal transduction, or c) IL2 from binding to an IL2Rβ/IL2Rγ complex and an IL15/IL15Rα complex from binding to an IL15Rβ/IL15Rγ complex, or any combination thereof.

Aspect 83: The method of any one of aspects 78-82, wherein the effective amount of the IL2R/IL15R inhibitor does not significantly disrupt IL2 from binding to a high affinity IL2Rα/IL2Rβ/IL2Rγ complex or does not significantly disrupt IL2 from signaling through a high affinity IL2Rα/IL2Rβ/IL2Rγ complex.

Aspect 84: The method of any one of aspects 78-83, wherein an IL2-stimulated Janus kinase (JAK) signaling pathway and an IL15-stimulated Janus kinase (JAK) signaling pathway are disrupted or inhibited in cells of the subject receiving the effective amount of the IL2R/IL15R inhibitor.

Aspect 85: The method of any one of aspects 78-84, wherein the IL2R/IL15R inhibitor is an antibody.

Aspect 86: The method of any one of aspects 79-85, wherein the antibody is an anti-CD122 antibody.

Aspect 87: The method of aspect 86, wherein the anti-CD122 antibody is a monoclonal antibody.

Aspect 88: The method of any one of aspects 79-87, wherein the antibody is a human antibody, a humanized antibody, or a chimeric antibody.

Aspect 89: The method of any one of aspects 79-88, wherein the antibody comprises an Immunoglobulin G (IgG) antibody or variant thereof.

Aspect 90: The method of aspect 89, wherein the IgG antibody or variant thereof comprises an IgG1 antibody or variant thereof, an IgG2 antibody or variant thereof, an IgG3 antibody or variant thereof, or an IgG4 antibody or variant thereof.

Aspect 91: The method of any one of aspects 79-90, wherein the antibody or its antigen-binding fragment thereof comprises IgG-scFv, IgA, IgM, IgE antibody, nanobody, mini-antibody, minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, or intrabody.

Aspect 92: The method of any one of aspects 86-91, wherein the anti-CD122 antibody interferes with IL15 binding to the IL15R and with IL2 binding to the IL2R.

Aspect 93: The method of any one of aspects 86-92, wherein the anti-CD122 antibody diminishes or disrupts IL2-induced signal transduction.

Aspect 94: The method of any one of aspects 86-93, wherein the anti-CD122 antibody diminishes or disrupts IL15-induced signal transduction.

Aspect 95: The method of any one of aspects 86-94, wherein the anti-CD122 antibody diminishes or disrupts IL2-induced signal transduction and IL15-induced signal transduction.

Aspect 96: The method of any one of aspects 78-95, wherein the administering an effective amount of the IL2R/IL15R inhibitor delays an onset of one or more symptoms of cGvHD in the subject.

Aspect 97: The method of any one of aspects 78-96, wherein the administering an effective amount of the IL2R/IL15R inhibitor alleviates one or more symptoms of cGvHD in the subject.

Aspect 98: The method of any one of aspects 78-97, wherein the administering an effective amount of the IL2R/IL15R inhibitor significantly relieves cGvHD severity in the subject.

Aspect 99: The method of any one of aspects 78-98, wherein the administering improves an objective response rate (ORR) at 6 months compared to a first-line standard of care therapy, a second-line standard of care therapy, or a third-line standard of care therapy.

Aspect 100: The method of aspect 99, wherein the first-line standard of care therapy comprises treatment with one or more corticosteroids.

Aspect 101: The method of aspect 99, wherein the first-line standard of care therapy comprises treatment with one or more JAK inhibitors.

Aspect 102: The method of aspect 99, wherein the first-line standard of care therapy comprises treatment with ruxolitinib.

Aspect 103: The method of any one of aspects 99-102, wherein the second-line standard of care therapy comprises treatment with either ruxolitinib or ibrutinib.

Aspect 104: The method of any one of aspects 99-103, wherein the second-line standard of care therapy comprises treatment with one or more corticosteroids.

Aspect 105: The method of any one of aspects 99-104, wherein the third-line standard of care therapy comprises treatment with belumosudil.

Aspect 106: The method of any one or aspects 96-105, wherein the one or more symptoms of cGvHD comprises skin rash, raised skin, discolored skin, itchy skin, thickened skin, tightened skin, damaged sweat glands, intolerance to temperature changes, abdominal swelling, yellow discoloration of the eyes, jaundice, elevated or abnormal liver enzyme levels in the blood, dry eyes, changes in vision, dry mouth, white patches in the oral cavity, painful mouth ulcers, pain or sensitivity to hot, cold, spicy, and/or acidic foods, pain or sensitivity to carbonated beverages, shortness of breath, dry cough, chronic cough, wheezing, difficulty breathing, pulmonary changes observed on a chest X-ray, difficulty swallowing, difficulty eating, pain with swallowing, gum disease, tooth decay, loss of appetite, weight loss, nausea, vomiting, diarrhea, stomach pain, fatigue, muscle weakness, muscle cramps, neuromuscular pain, decreased range of motion in joints, decreased range of extension of fingers, wrists, elbows, knees, and/or ankles, tightness in joints or in connective tissue, change in physical activity level, change in locomotor activity level, change in posture, change in gait, vaginal dryness, vaginal itching, vaginal pain, vaginal ulcerations and scarring, narrowing of the vagina. painful vaginal intercourse, narrowing and/or scaring of the urethra, itching and/or scarring of the penis and scrotum, irritation of the penis, change in skin texture, change in skin integrity, scaling of skin, areas of denuded skin, loss of hair on the head, hard nails, brittle nails, nail loss, changes in nail texture, premature graving of the hair, or changes in hair texture, or any combination thereof.

Aspect 107: The method of any one of aspects 78-106, wherein the administering an effective amount of the IL2R/IL15R inhibitor reduces expression of one or more biomarkers of cGvHD in the subject.

Aspect 108: The method of any one of aspects 78-107, wherein the administering an effective amount of the IL2R/IL15R inhibitor increases the survival rate of the subject.

Aspect 109: The method of any one of aspects 78-108, wherein the administering an effective amount of the IL2R/IL15R inhibitor decreases a risk of cGvHD-symptom relapse in the subject.

Aspect 110: The method of any one of aspects 78-109, wherein the IL2R/IL15R inhibitor is administered systemically, locally, intradermally, subcutaneously, or topically.

Aspect 111: The method of aspect 110, wherein the IL2R/IL15R inhibitor administered systemically is administered by intravenous injection, by enteral administration, or through inhalation.

Aspect 112: The method of aspect 111, wherein the IL2R/IL15R inhibitor administered by enteral administration is administered orally.

Aspect 113: A method of preventing or treating GvHD in a subject in need thereof, the method comprising: administering to the subject an effective amount of i) an IL2R/IL15R inhibitor and ii) a JAK inhibitor, thereby preventing or treating GvHD in the subject.

Aspect 114: The method of aspect 113, wherein the GvHD is acute graft versus host disease (aGvHD).

Aspect 115: The method of aspect 114, wherein the aGvHD is steroid refractory aGvHD.

Aspect 116: The method of aspect 114 or 115, wherein the aGvHD is JAK inhibitor refractory aGvHD.

Aspect 117: The method of aspect 114 or 115, wherein the aGvHD is ruxolitinib refractory aGvHD.

Aspect 118: The method of aspect 113, wherein the GvHD is chronic graft versus host disease (cGvHD).

Aspect 119: The method of any one of aspects 113-118, wherein the IL2R/IL15R inhibitor and the JAK inhibitor are co-administered.

Aspect 120: The method of any one of aspects 113-119, wherein the IL2R/IL15R inhibitor and the JAK inhibitor are administered separately or sequentially.

Aspect 121: The method of any one of aspects 113-120, wherein the JAK inhibitor is selected from the group consisting of ruxolitinib, abrocitinib, baricitinib, delgocitinib, fedratinib, filgotinib, oclacitinib, pacritinib, peficitinib, tofacitinib, itacitinib and upadacitinib.

Aspect 122: The method of any one of aspects 113-121, wherein the JAK inhibitor is ruxolitinib.

Aspect 123: The method of any one of aspects 113-122, wherein the IL2R/IL15R inhibitor is a CD122 inhibitor.

Aspect 124: The method of any one of aspects 113-123, wherein the IL2R/IL15R inhibitor is an antibody or its antigen-binding fragment thereof, a small molecule inhibitor, a peptide inhibitor, or a nucleotide-based inhibitor.

Aspect 125: The method of aspect 124, wherein the antibody is a human antibody, a humanized antibody, or a chimeric antibody.

Aspect 126: The method of aspect 124 or 125, wherein the antibody comprises an Immunoglobulin G (IgG) antibody or variant thereof.

Aspect 127: The method of aspect 126, wherein the IgG antibody or variant thereof comprises an IgG1 antibody or variant thereof, an IgG2 antibody or variant thereof, an IgG3 antibody or variant thereof, or an IgG4 antibody or variant thereof.

Aspect 128: The method of aspect 113 or 114, wherein the antibody or its antigen-binding fragment thereof comprises IgG-scFv, IgA, IgM, IgE antibody, nanobody, mini-antibody, minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, or intrabody.

Aspect 129: The method of any one of aspects 113-128, wherein the IL2R/IL15 receptor inhibitor is an anti-CD122 antibody.

Aspect 130: The method of any one of aspects 113-129, wherein the JAK inhibitor is administered first and the IL2R/IL15R inhibitor is administered second.

Aspect 131: The method of any one of aspects 113-130, wherein the administering an effective amount of the IL2R/IL15R inhibitor and the JAK inhibitor delays an onset of one or more symptoms of aGvHD or cGvHD in the subject.

Aspect 132: The method of any one of aspects 113-131, wherein the administering an effective amount of the IL2R/IL15R inhibitor and the JAK inhibitor alleviates one or more symptoms of aGvHD or cGvHD in the subject.

Aspect 133: The method of aspect 131 or 132, wherein the one or more symptoms of aGvHD comprises itchy skin, skin rash, reddened patches on the skin, yellow discoloration of the skin, blisters on the skin, exposed surfaces of the skin flaking off, yellow discoloration of the eyes, jaundice, elevated liver enzyme levels in the blood, nausea, vomiting, diarrhea, abdominal cramping, loss of appetite, or weight loss, or any combination thereof.

Aspect 134: The method of aspect 131 or 132, wherein the one or more symptoms of cGvHD comprises skin rash, raised skin, discolored skin, itchy skin, thickened skin, tightened skin, damaged sweat glands, intolerance to temperature changes, abdominal swelling, yellow discoloration of the eyes, jaundice, elevated or abnormal liver enzyme levels in the blood, dry eyes, changes in vision, dry mouth, white patches in the oral cavity, painful mouth ulcers, pain or sensitivity to hot, cold, spicy, and/or acidic foods, pain or sensitivity to carbonated beverages, shortness of breath, dry cough, chronic cough, wheezing, difficulty breathing, pulmonary changes observed on a chest X-ray, difficulty swallowing, difficulty eating, pain with swallowing, gum disease, tooth decay, loss of appetite, weight loss, nausea, vomiting, diarrhea, stomach pain, fatigue, muscle weakness, muscle cramps, neuromuscular pain, decreased range of motion in joints, decreased range of extension of fingers, wrists, elbows, knees, and/or ankles, tightness in joints or in connective tissue, change in physical activity level, change in locomotor activity level, change in posture, change in gait, vaginal dryness, vaginal itching, vaginal pain, vaginal ulcerations and scarring, narrowing of the vagina, painful vaginal intercourse, narrowing and/or scaring of the urethra, itching and/or scarring of the penis and scrotum, irritation of the penis, change in skin texture, change in skin integrity, scaling of skin, areas of denuded skin, loss of hair on the head, hard nails, brittle nails, nail loss, changes in nail texture, premature graying of the hair, or changes in hair texture, or any combination thereof.

Aspect 135: The method of any one of aspects 113-134, wherein the administering an effective amount of the IL2R/IL15R inhibitor and the JAK inhibitor reduces expression of one or more biomarkers of aGvHD or cGvHD in the subject.

Aspect 136: The method of any one of aspects 113-135, wherein the administering an effective amount of the IL2R/IL15R inhibitor and the JAK inhibitor increases the survival rate of the subject.

Aspect 137: The method of any one of aspects 113-136, wherein the administering an effective amount of the IL2R/IL15R inhibitor and the JAK inhibitor increases the survival rate of the subject compared with a subject treated with a JAK inhibitor as a monotherapy.

Aspect 138: The method of any one of aspects 113-137, wherein the administering an effective amount of the IL2R/IL15R inhibitor and the JAK inhibitor decreases a risk of aGvHD-symptom relapse or cGvHD-symptom relapse.

Aspect 139: The method of any one of aspects 113-138, wherein the IL2R/IL15R inhibitor and the JAK inhibitor are administered by the same route of administration.

Aspect 140: The method of any one of aspects 113-139, wherein the IL2R/IL15R inhibitor and the JAK inhibitor are administered by separate routes of administration.

Aspect 141: The method of any one of aspects 113-140, wherein the IL2R/IL15R inhibitor is administered systemically, locally, intradermally, subcutaneously, or topically, and wherein the JAK inhibitor is administered systemically, locally, intradermally, subcutaneously, or topically.

Aspect 142: The method of aspect 141, wherein the IL2R/IL15R inhibitor administered systemically is administered by intravenous injection, by subcutaneous injection, by enteral administration, or through inhalation.

Aspect 143: The method of aspect 141 or 142, wherein the JAK inhibitor administered systemically is administered by intravenous injection, by subcutaneous injection, by enteral administration, or through inhalation.

Aspect 144: The method of aspect 142 or 143, wherein the IL2R/IL15R inhibitor administered by enteral administration is administered orally.

Aspect 145: The method of aspect 143 or 144, wherein the JAK inhibitor administered by enteral administration is administered orally.

Aspect 146: The method of any one of aspects 113-145, wherein the administering an effective amount of the IL2R/IL15R inhibitor and the JAK inhibitor significantly prevents development of an extent of cGvHD severity in the subject.

Aspect 147: The method of any one of aspects 113-146, wherein the administering an effective amount of the IL2R/IL15R inhibitor and the JAK inhibitor significantly relieves GvHD severity.

Aspect 148: The method of aspect 147, wherein the administering improves an ORR at 28 days compared to treatment comprising ruxolitinib monotherapy.

Aspect 149: The method of any one of aspects 113-148, wherein the administering significantly prevents development of an extent of cGvHD severity in the subject according to a total cGvHD assessment compared to placebo treatment, a first-line standard of care cGvHD therapy, a second-line standard of care cGvHD therapy, or a third-line standard of care cGvHD therapy.

Aspect 150: The method of aspect 149, wherein the first-line standard of care therapy comprises treatment with one or more corticosteroids.

Aspect 151: The method of aspect 149, wherein the first-line standard of care therapy comprises treatment with one or more JAK inhibitors.

Aspect 152: The method of aspect 149, wherein the first-line standard of care therapy comprises treatment with ruxolitinib.

Aspect 153: The method of any one of aspects 149-152, wherein the second-line standard of care therapy comprises treatment with either ruxolitinib or ibrutinib.

Aspect 154: The method of any one of aspects 149-153, wherein the second-line standard of care therapy comprises treatment with one or more corticosteroids.

Aspect 155: The method of any one of aspects 149-154, wherein the third-line standard of care therapy comprises treatment with belumosudil.

Aspect 156: The method of any one of aspects 113-155, wherein the administering significantly prevents development of an extent of cGvHD severity in the subject according to a total cGvHD assessment compared to treatment comprising ruxolitinib monotherapy.

Aspect 157: The method of any one of aspects 1-156, wherein the effective amount of the IL2R inhibitor, the IL15R inhibitor, or the IL2R/IL15R inhibitor is a therapeutically effective amount to treat one of more symptoms of aGvHD or cGvHD in the subject.

Aspect 158: The method of any one of aspects 34-77 or 113-157, wherein the effective amount of the JAK inhibitor is a therapeutically effective amount to treat one of more symptoms of aGvHD or cGvHD in the subject.

What is claimed is:

1. A method of preventing or treating chronic graft versus host disease (cGvHD) in a subject in need thereof, the method comprising: administering to the subject an effective amount of an IL2 receptor (IL2R) inhibitor, thereby preventing or treating cGvHD in the subject.

2. The method of claim 1, wherein the IL2R inhibitor functions as an IL2R/IL15R inhibitor.

3. A method of preventing or treating chronic graft versus host disease (cGvHD) in a subject in need thereof, the method comprising: administering to the subject an effective amount of an IL15 receptor (IL15R) inhibitor, thereby preventing or treating cGvHD in the subject.

4. The method of claim 3, wherein the IL15R inhibitor functions as an IL2R/IL15R inhibitor.

5. The method of claim 1 or 2, wherein the IL2R inhibitor is a small molecule inhibitor, an antibody or its antigen-binding fragment thereof, a peptide inhibitor, or nucleotide-based inhibitor.

6. The method of claim 3 or 4, wherein the IL15R inhibitor is a small molecule inhibitor, an antibody or its antigen-binding fragment thereof, a peptide inhibitor, or nucleotide-based inhibitor.

7. The method of any one of claim 1-2 or 5, wherein the IL2R inhibitor is a CD122 inhibitor

8. The method of any one of claim 3-4 or 6, wherein the IL15R inhibitor is a CD122 inhibitor.

9. The method of any one of claim 1-2, 5, or 7, wherein the IL2R inhibitor interferes with an IL2 binding to the IL2R.

10. The method of any one of claim 1-2, 5, 7, or 9, wherein the IL2R inhibitor disrupts or diminishes: a) an IL2 from binding to an IL2Rβ/IL2Rγ complex, b) IL2-induced signal transduction, or c) CD122-mediated signal transduction, or a combination thereof.

11. The method of any one of claim 3-4, 6, or 8, wherein the IL15R inhibitor interferes with an IL15 binding to the IL15R.

12. The method of any one of claim 3-4, 6, or 11, wherein the IL15R inhibitor disrupts or diminishes: a) an IL15/IL15Rα complex from binding to an IL15Rβ/IL15Rγ complex, b) IL15-induced signal transduction, or c) CD122-mediated signal transduction, or a combination thereof.

13. The method of any one of claims 1-12, wherein the effective amount of the IL2R inhibitor, the IL15R inhibitor, or the IL2R/IL15R inhibitor does not significantly disrupt IL2 from: a) binding to a high affinity IL2Rα/IL2Rβ/IL2Rγ complex, or b) signaling through a high affinity IL2Rα/IL2Rβ/IL2Rγ complex.

14. The method of any one of claim 1-2, 5, 7, 9-10, or 13, wherein an IL2-stimulated Janus kinase (JAK) signaling pathway is disrupted or inhibited in cells of the subject receiving the effective amount of the IL2R inhibitor.

15. The method of any one of claim 3-4, 6, 8, or 11-13, wherein an IL15-stimulated Janus kinase (JAK) signaling pathway is disrupted or inhibited in cells of the subject receiving the effective amount of the IL15R inhibitor.

16. The method of any one of claims 1-15, wherein the IL2R inhibitor is an antibody.

17. The method of any one of claims 1-15, wherein the IL15R inhibitor is an antibody.

18. The method of any one of claims 1-17, wherein the IL2R/IL15R inhibitor is an antibody.

19. The method of any one of claims 5-18, wherein the antibody is an anti-CD122 antibody.

20. The method of claim 19, wherein the anti-CD122 antibody is a monoclonal antibody.

21. The method of any one of claims 5-20, wherein the antibody is a human antibody, a humanized antibody, or a chimeric antibody.

22. The method of any one of claims 5-21, wherein the antibody comprises an Immunoglobulin G (IgG) antibody or variant thereof.

23. The method of claim 22, wherein the IgG antibody or variant thereof comprises an IgG1 antibody or variant thereof, an IgG2 antibody or variant thereof, an IgG3 antibody or variant thereof, or an IgG4 antibody or variant thereof.

24. The method of any one of claims 5-23, wherein the antibody or its antigen-binding fragment thereof comprises IgG-scFv, IgA, IgM, IgE antibody, nanobody, mini-antibody, minibody, scFv-CH3 KIH, Fab-scFy-Fc KIH, Fab-scFv, scFv-CH-CL-scFv, Fab′, F(ab′)2, F(ab′)3, F(ab′)2-scFv2, scFv, scFv-KIH, Fab-scFv-Fc, or intrabody.

25. The method of any one of claims 20-24, wherein the anti-CD122 antibody interferes with: i) IL15 binding to the IL15R, ii) IL2 binding to the IL2R, or iii) IL15 binding to the IL15R and IL2 binding to the IL2R.

26. The method of any one of claims 20-25, wherein the anti-CD122 antibody diminishes or disrupts IL2-induced signal transduction, IL15-induced signal transduction, or IL2-induced signal transduction and IL15-induced signal transduction.

27. The method of any one of claims 1-26, wherein the administering an effective amount of the IL2R inhibitor or the IL15R inhibitor: a) delays an onset of one or more symptoms of cGvHD in the subject, b) alleviates one or more symptoms of cGvHD in the subject, or c) significantly relieves cGvHD severity in the subject, or any combination thereof.

28. The method of any one of claims 1-27, wherein the administering improves an objective response rate (ORR) at 6 months compared to a first-line standard of care therapy, a second-line standard of care therapy, or a third-line standard of care therapy.

29. The method of claim 28, wherein the first-line standard of care therapy comprises treatment with one or more corticosteroids or treatment with one or more JAK inhibitors.

30. The method of claim 28, wherein the first-line standard of care therapy comprises treatment with ruxolitinib.

31. The method of any one of claims 28-30, wherein the second-line standard of care therapy comprises treatment with either ruxolitinib or ibrutinib.

32. The method of any one of claims 28-31, wherein the second-line standard of care therapy comprises treatment with one or more corticosteroids.

33. The method of any one of claims 28-32, wherein the third-line standard of care therapy comprises treatment with belumosudil.

34. The method of any one or claims 27-33, wherein the one or more symptoms of cGvHD comprises skin rash, raised skin, discolored skin, itchy skin, thickened skin, tightened skin, damaged sweat glands, intolerance to temperature changes, abdominal swelling, yellow discoloration of the eyes, jaundice, elevated or abnormal liver enzyme levels in the blood, dry eyes, changes in vision, dry mouth, white patches in the oral cavity, painful mouth ulcers, pain or sensitivity to hot, cold, spicy, and/or acidic foods, pain or sensitivity to carbonated beverages, shortness of breath, dry cough, chronic cough, wheezing, difficulty breathing, pulmonary changes observed on a chest X-ray, difficulty swallowing, difficulty eating, pain with swallowing, gum disease, tooth decay, loss of appetite, weight loss, nausea, vomiting, diarrhea, stomach pain, fatigue, muscle weakness, muscle cramps, neuromuscular pain, decreased range of motion in joints, decreased range of extension of fingers, wrists, elbows, knees, and/or ankles, tightness in joints or in connective tissue, change in physical activity level, change in locomotor activity level, change in posture, change in gait, vaginal dryness, vaginal itching, vaginal pain, vaginal ulcerations and scarring, narrowing of the vagina, painful vaginal intercourse, narrowing and/or scaring of the urethra, itching and/or scarring of the penis and scrotum, irritation of the penis, change in skin texture, change in skin integrity, scaling of skin, areas of denuded skin, loss of hair on the head, hard nails, brittle nails, nail loss, changes in nail texture, premature graving of the hair, or changes in hair texture, or any combination thereof.

35. The method of any one of claims 1-34, wherein the administering an effective amount of the IL2R inhibitor or IL15R inhibitor: a) reduces expression of one or more biomarkers of cGvHD in the subject, b) increases the survival rate of the subject, or c) decreases a risk of cGvHD-symptom relapse in the subject, or any combination thereof.

36. The method of any one of claims 1-35, wherein the IL2R inhibitor or IL15R inhibitor is administered systemically, locally, intradermally, subcutaneously, or topically.

37. The method of claim 36, wherein the IL2R inhibitor or IL15R inhibitor administered systemically is administered by intravenous injection, by enteral administration, or through inhalation.

38. The method of claim 37, wherein the IL2R inhibitor or IL15R inhibitor administered by enteral administration is administered orally.

39. A method of preventing or treating GvHD in a subject in need thereof, the method comprising: administering to the subject an effective amount of i) an IL15 receptor (IL15R) inhibitor and ii) a JAK inhibitor, thereby preventing or treating GvHD in the subject.

40. A method of preventing or treating GvHD in a subject in need thereof, the method comprising: administering to the subject an effective amount of i) an IL2 receptor (IL2R) inhibitor and ii) a JAK inhibitor, thereby preventing or treating GvHD in the subject.

41. The method of claim 39 or 40, wherein the IL15R inhibitor or the IL2R inhibitor functions as an IL2R/IL15R inhibitor.

42. The method of any one of claims 39-41, wherein the GvHD is acute graft versus host disease (aGvHD).

43. The method of claim 42, wherein the aGvHD is steroid refractory aGvHD or JAK inhibitor refractory aGvHD.

44. The method of claim 42, wherein the aGvHD is ruxolitinib refractory aGvHD.

45. The method of any one of claims 39-41, wherein the GvHD is chronic graft versus host disease (cGvHD).

46. The method of any one of claims 39-45, wherein the IL15R inhibitor and the JAK inhibitor are co-administered or the IL2R inhibitor and the JAK inhibitor are co-administered.

47. The method of any one of claims 39-46, wherein the co-administered IL15R inhibitor and JAK inhibitor or the co-administered IL2R inhibitor and JAK inhibitor are administered separately or sequentially.

48. The method of any one of claims 39-47, wherein the JAK inhibitor is selected from the group consisting of ruxolitinib, abrocitinib, baricitinib, delgocitinib, fedratinib, filgotinib, oclacitinib, pacritinib, peficitinib, tofacitinib, itacitinib and upadacitinib.

49. The method of any one of claims 39-48, wherein the JAK inhibitor is ruxolitinib.

50. The method of any one of claims 39-49, wherein the IL15R inhibitor or the IL2R inhibitor is a CD122 inhibitor.

51. The method of any one of claims 39-50, wherein the IL15R inhibitor or the IL2R inhibitor is an antibody or its antigen-binding fragment thereof, a small molecule inhibitor, a peptide inhibitor, or a nucleotide-based inhibitor.

52. The method of claim 51, wherein the antibody is a human antibody, a humanized antibody, or a chimeric antibody.

53. The method of claim 51 or 52, wherein the antibody comprises an Immunoglobulin G (IgG) antibody or variant thereof.

54. The method of claim 53, wherein the IgG antibody or variant thereof comprises an IgG1 antibody or variant thereof, an IgG2 antibody or variant thereof, an IgG3 antibody or variant thereof, or an IgG4 antibody or variant thereof.

55. The method of any one of claims 51-54, wherein the antibody or its antigen-binding fragment thereof comprises IgG-scFv, IgA, IgM, IgE antibody, nanobody, mini-antibody, minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, or intrabody.

56. The method of any one of claims 39-55, wherein the IL15 receptor inhibitor or the IL2 receptor inhibitor is an anti-CD122 antibody.

57. The method of any one of claims 39-56, wherein the JAK inhibitor is administered first and the IL15R inhibitor or the IL2R inhibitor is administered second.

58. The method of any one of claims 39-57, wherein the administering an effective amount of the IL15R inhibitor and JAK inhibitor or the IL2R inhibitor and JAK inhibitor delays an onset of one or more symptoms of aGvHD or cGvHD in the subject or alleviates one or more symptoms of aGvHD or cGvHD in the subject.

59. The method of claim 58, wherein the one or more symptoms of aGvHD comprises itchy skin, skin rash, reddened patches on the skin, yellow discoloration of the skin, blisters on the skin, exposed surfaces of the skin flaking off, yellow discoloration of the eyes, jaundice, elevated liver enzyme levels in the blood, nausea, vomiting, diarrhea, abdominal cramping, loss of appetite, or weight loss, or any combination thereof.

60. The method of claim 58, wherein the one or more symptoms of cGvHD comprises skin rash, raised skin, discolored skin, itchy skin, thickened skin, tightened skin, damaged sweat glands, intolerance to temperature changes, abdominal swelling, yellow discoloration of the eyes, jaundice, elevated or abnormal liver enzyme levels in the blood, dry eyes, changes in vision, dry mouth, white patches in the oral cavity: painful mouth ulcers, pain or sensitivity to hot, cold, spicy, and/or acidic foods, pain or sensitivity to carbonated beverages, shortness of breath, dry cough, chronic cough, wheezing, difficulty breathing, pulmonary changes observed on a chest X-ray, difficulty swallowing, difficulty eating, pain with swallowing. gum disease, tooth decay, loss of appetite, weight loss, nausea, vomiting, diarrhea, stomach pain, fatigue, muscle weakness, muscle cramps, neuromuscular pain, decreased range of motion in joints, decreased range of extension of fingers, wrists, elbows, knees, and/or ankles, tightness in joints or in connective tissue, change in physical activity level, change in locomotor activity level, change in posture, change in gait, vaginal dryness, vaginal itching, vaginal pain, vaginal ulcerations and scarring, narrowing of the vagina, painful vaginal intercourse, narrowing and/or scaring of the urethra, itching and/or scarring of the penis and scrotum, irritation of the penis, change in skin texture, change in skin integrity; scaling of skin, areas of denuded skin, loss of hair on the head, hard nails, brittle nails, nail loss, changes in nail texture, premature graving of the hair, or changes in hair texture, or any combination thereof.

61. The method of any one of claims 39-60, wherein the administering an effective amount of the IL15R inhibitor and JAK inhibitor or the IL2R inhibitor and JAK inhibitor: a) reduces expression of one or more biomarkers of aGvHD or cGvHD in the subject, b) increases the survival rate of the subject, c) increases the survival rate of the subject compared with a subject treated with a JAK inhibitor as a monotherapy, or d) decreases a risk of aGvHD-symptom relapse or cGvHD-symptom relapse in the subject, or any combination thereof.

62. The method of any one of claims 39-61, wherein the IL15R inhibitor and JAK inhibitor or the IL2R inhibitor and JAK inhibitor are administered by the same route of administration.

63. The method of any one of claims 39-61, wherein the IL15R inhibitor and JAK inhibitor or the IL2R inhibitor and JAK inhibitor are administered by separate routes of administration.

64. The method of any one of claims 39-63, wherein the IL15R inhibitor or the IL2R inhibitor is administered systemically, locally, intradermally, subcutaneously, or topically, and wherein the JAK inhibitor is administered systemically, locally, intradermally, subcutaneously, or topically.

65. The method of claim 64, wherein the IL15R inhibitor or IL2R inhibitor administered systemically is administered by intravenous injection, by subcutaneous injection, by enteral administration, or through inhalation.

66. The method of claim 64 or 65, wherein the JAK inhibitor administered systemically is administered by intravenous injection, by subcutaneous injection, by enteral administration, or through inhalation.

67. The method of claim 65 or 66, wherein the IL15R inhibitor or IL2R inhibitor administered by enteral administration is administered orally.

68. The method of claim 66 or 67, wherein the JAK inhibitor administered by enteral administration is administered orally.

69. The method of any one of claims 45-68, wherein the administering an effective amount of the IL15R inhibitor and JAK inhibitor or the IL2R inhibitor and JAK inhibitor significantly prevents development of an extent of cGvHD severity in the subject or significantly relieves GvHD severity.

70. The method of claim 69, wherein the administering improves an ORR at 28 days compared to treatment comprising ruxolitinib monotherapy.

71. The method of any one of claims 45-70, wherein the administering significantly prevents development of an extent of cGvHD severity in the subject according to a total cGvHD assessment compared to placebo treatment, a first-line standard of care cGvHD therapy, a second-line standard of care cGvHD therapy, or a third-line standard of care cGvHD therapy.

72. The method of claim 71, wherein the first-line standard of care therapy comprises treatment with one or more corticosteroids or treatment with one or more JAK inhibitors.

73. The method of claim 71, wherein the first-line standard of care therapy comprises treatment with ruxolitinib.

74. The method of claim any one of claims 71-73, wherein the second-line standard of care therapy comprises treatment with either ruxolitinib or ibrutinib.

75. The method of any one of claims 71-74, wherein the second-line standard of care therapy comprises treatment with one or more corticosteroids.

76. The method of any one of claims 71-75, wherein the third-line standard of care therapy comprises treatment with belumosudil.

77. The method of any one of claims 39-76, wherein the administering significantly prevents development of an extent of cGvHD severity in the subject according to a total cGvHD assessment compared to treatment comprising ruxolitinib monotherapy.

78. A method of preventing or treating chronic graft versus host disease (cGvHD) in a subject in need thereof, the method comprising: administering to the subject an effective amount of an IL2R/IL15R inhibitor, thereby preventing or treating cGvHD in the subject.

79. A method of preventing or treating chronic graft versus host disease (cGvHD) in a subject in need thereof, the method comprising: administering to the subject an effective amount of: i) an IL2R inhibitor, ii) an IL15R inhibitor, or iii) an IL2R/IL15R inhibitor, thereby preventing or treating cGvHD in the subject.

80. A method of preventing or treating GvHD in a subject in need thereof, the method comprising: administering to the subject an effective amount of i) an IL2 receptor (IL2R) inhibitor, an IL15 receptor (IL15R) inhibitor, or an IL2R/IL15R inhibitor, and ii) a JAK inhibitor, thereby preventing or treating GvHD in the subject.

81. The method of any one of claims 1-80, wherein the effective amount of the IL2R inhibitor, the IL15R inhibitor, or the IL2R/IL15R inhibitor is a therapeutically effective amount to treat one of more symptoms of aGvHD or cGvHD in the subject.
